(19) United States
(12) Patent Application Publication
(10) Pub. No.: US 2016/0361357 A1
(43) Pub. Date: Dec. 15, 2016

(54) STABLE HIGH CONCENTRATION STRONTIUM COMPOUNDS AND FORMULATIONS FOR TOPICAL APPLICATION

(71) Applicant: Cosmederm Bioscience, Inc., San Diego, CA (US)

(72) Inventor: Gary S. Hahn, San Diego, CA (US)

(21) Appl. No.: 15/249,293

(22) Filed: Aug. 26, 2016

Related U.S. Application Data
(63) Continuation of application No. PCT/US2016/017364, filed on Feb. 10, 2016.
(60) Provisional application No. 62/115,054, filed on Feb. 11, 2015.

(51) Int. Cl. A61K 33/14; A61K 9/107; A61K 9/00; A61K 33/00; A61K 47/32

(52) U.S. Cl. CPC A61K 33/14; A61K 33/00; A61K 47/32; A61K 9/0014; A61K 9/107

(57) ABSTRACT

The disclosure herein relates to high concentration strontium compounds. The high concentrations are achievable through the use of a specialized polymer. More specifically, the polymers form a three dimensional structure having a hydrophilic interior and hydrophobic exterior.

### INCORPORATION BY REFERENCE TO RELATED APPLICATIONS

Any and all priority claims identified in the Application Data Sheet, or any correction thereto, are hereby incorporated by reference under 37 CFR 1.57. This application is a continuation, under 35 U.S.C. §120, of International Patent Application No. PCT/US2016/017364, filed on Feb. 10, 2016 under the Patent Cooperation Treaty (PCT), which was published by the International Bureau in English on Aug. 18, 2016, which designates the United States and claims the benefit of U.S. Provisional Application No. 62/115,054, filed Feb. 11, 2015. Each of the aforementioned applications is incorporated by reference herein in its entirety, and each is hereby expressly made a part of this specification.

### TECHNICAL FIELD

Topical formulations having a high concentration of strontium are provided. The high strontium concentrations are achievable through, for example, the use of a specialized polymer.

### BACKGROUND

Topically-applied strontium, in divalent ionic form, has the ability to rapidly suppress acute sensory irritation (e.g., stinging, burning pain and/or itching) and accompanying inflammation due to chemical irritants, electromagnetic radiation, “environmental irritants” and diseases. While not being bound or otherwise limited by any particular biochemical mechanism, it has been theorized that strontium's anti-irritant activity was due to the ability of strontium to selectively suppress activation of Type C Nociceptors (TCN), the only sensory nerves that produce and transmit stinging, burning, pain, and itching sensations and the neurogenic inflammatory response that can accompany TCN activation.

When compared to the existing topical drugs able to suppress such sensory irritation like lidocaine or NOVOCAIN®, the local anesthetic typically used by dentists during dental procedures, strontium has a unique property—it is highly selective for only the TCN and does not significantly affect the many other sensory nerves that provide normal tactile sensations and “cutaneous awareness.” Since lidocaine and other topical local anesthetics lack this specificity for TCN, they can cause numbness and loss of function.

Current strontium formulations have been limited in the amount of strontium that can be added to a topically applied formula such as a lotion, cream, or gel. The inventors have found that high concentrations of strontium salts disrupt emulsions or cause ingredients to precipitate resulting in topical products with a short shelf-life. Additionally, inventors have found that as the strontium concentrations increase, the tackiness of the topical formula increases resulting in a product that is difficult to apply to the skin and uncomfortable to the user. Lastly, inventors have found that high concentrations of strontium can cause substantial stinging and burning when applied to abraded or inflamed skin. Therefore, it is desirable to create high concentration strontium formulations that have a long shelf-life, are easily spread and absorbed into the skin of the user, and do not cause osmotic shock when applied to the skin.

### SUMMARY

The following simplified summary provides a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented below.

In one embodiment, the disclosure herein relates to compositions and formulations comprising a strontium moiety and a polyacrylate crosspolymer-6 polymer. The compositions and formulations include high concentrations of the strontium moiety, such as concentrations of at least 5% of the total weight of the composition or formulation. Alternatively the concentration of the strontium moiety can be at least 8% of the total weight of the composition or formulation. Or, the concentration of the strontium moiety can be at least 10% of the total weight of the composition or formulation. Or the concentration of the strontium moiety can be at least 12% of the total weight of the composition or formulation. Or the concentration of the strontium moiety can be at least 15% of the total weight of the composition or formulation

In another embodiment, the disclosure herein relates to compositions and formulations comprising elemental strontium and a polyacrylate crosspolymer-6 polymer. The compositions and formulations include high concentrations of the elemental strontium, such as concentrations of at least 5% of the total weight of the composition or formulation. Alternatively the concentration of the elemental strontium can be at least 8% of the total weight of the composition or formulation. Or, the concentration of the elemental strontium can be at least 10% of the total weight of the composition or formulation. Or the concentration of the elemental strontium can be at least 12% of the total weight of the composition or formulation. Or the concentration of the elemental strontium can be at least 15% of the total weight of the composition or formulation.

In another embodiment, the compositions and formulations described herein include the polymer at a concentration of less than 3% of the total weight of the composition or formulation. Alternatively, the concentration of the polymer can be less than 2% of the total weight of the composition or formulation. Or the concentration of the polymer can be less than 1% of the total weight of the composition or formulation. Or the concentration of the polymer is, or is about, 0.5% of the total weight of the composition or formulation. The compositions and formulations described herein can have a pH of less than 5. Alternatively, the pH can be less than 4. Or the pH can be less than 3.

In yet another embodiment, the compositions and formulations described herein can have a viscosity of less than 8,000 centipoise. Alternatively, the viscosity can be less than 5,000 centipoise. Or the viscosity can be less than 3,000 centipoise. When a viscosity is referred to in units of centipoise, it is measured at 20° C., unless otherwise stated.

In another embodiment, in the compositions and formulations described herein, the strontium moiety can be a strontium salt. Often the strontium salt can be strontium nitrate, strontium chloride, strontium chloride hexahydrate, strontium sulfate, strontium carbonate, strontium hydroxide, strontium hydrosulfide, strontium oxide, strontium acetate, strontium mono-carboxylic acid, strontium di-carboxylic acid, strontium tri-carboxylic acid, strontium quatro-carboxylic acid, strontium amino carboxylic acid, strontium glutamate, strontium aspartate, strontium malonate, strontium maleate, strontium citrate, strontium threonate, strontium lactate, strontium pyruvate, strontium ascorbate, strontium alpha-ketoglutarate, or strontium succinate. In one embodiment, the strontium salt is strontium nitrate. In another embodiment, the strontium salt is strontium chloride. In another embodiment, the strontium salt is strontium hydroxide.

In another embodiment, in the compositions and formulations described herein, the elemental strontium can be from a strontium salt. Often the strontium salt can be strontium nitrate, strontium chloride, strontium chloride hexahydrate, strontium sulfate, strontium carbonate, strontium hydroxide, strontium hydrosulfide, strontium oxide, strontium acetate, strontium mono-carboxylic acid, strontium di-carboxylic acid, strontium tri-carboxylic acid, strontium quatro-carboxylic acid, strontium amino carboxylic acid, strontium glutamate, strontium aspartate, strontium malonate, strontium maleate, strontium citrate, strontium threonate, strontium lactate, strontium pyruvate, strontium ascorbate, strontium alpha-ketoglutarate, or strontium succinate. In one embodiment, the strontium salt is strontium nitrate. In another embodiment, the strontium salt is strontium chloride. In another embodiment, the strontium salt is strontium hydroxide.

Frequently, the compositions and formulations described herein can further include at least one active agent. In one embodiment, the active agent can be analgesics, corticosteroids, sunscreen, vitamins, insect repellent, humectants, moisturizers, soothing agents, or penetration enhancers.

Often, the compositions and formulations described herein can further include at least one excipient. In one embodiment, the excipient can be solvents, emulsifying agents, dispersants, thickeners, wetting agents, surfactants, foaming agents, defoaming agents, lubricants, evaporation reducers, preservatives, stabilizers, antioxidants, antimicrobials, reducing agents, fragrance or color.

Frequently, the compositions and formulations described herein can be made into topically applied compositions and formulations. Such topical compositions and formulations can be in the form of an emulsion such as a lotion, cream, or gel.

Another embodiment is a method of manufacturing the compositions and formulations described herein. The manufacturing involves (a) wetting the polyacrylate crosspolymer-6 polymer; (b) dissolving the strontium moiety into an aqueous phase; and (c) combining the products of step (a) and step (b) to create an emulsion. In one embodiment, the polymer is wetted in oil.

Pharmaceutical excipients can advantageously be used in the topical formulations or added to the wetted polymer. Excipients include, e.g., solvents, emollients and/or emulsifiers, oil bases, preservatives, antioxidants, tonicity adjusters, penetration enhancers and solubilizers, chelating agents, buffering agents, humectants, thickeners, surfactants, and combinations thereof.

Hydrophilic solvents or carriers include water; ethyl alcohol; isopropyl alcohol; mixtures of water and ethyl and/or isopropyl alcohols; glycerin; ethylene, propylene or butylene glycols; DMSO; and mixtures thereof. Hydrophobic solvents or carriers include mineral oils, vegetable oils, and silicone oils. If desired, components may be dissolved or dispersed in a hydrophobic oil phase, and the oil phase may then be emulsified in an aqueous phase comprising water, alone or in combination with lower alcohols, glycerin, and/or glycols.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is readily and economically available and is easy to work with. Other thickening agents include, for example, xanthan gum, carboxymethyl cellulose, hydroxypropyl cellulose, carbomer, and the like. The concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver active ingredients as described herein.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), Tocopherol, thimerosal, chlorobutanol, or the like, and mixtures thereof, can be employed. If a preservative is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The formulation can have a pH of between about 2.0 or less and 5.0 or more. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Other buffers include citric acid/sodium citrate, and dibasic sodium phosphate/citric acid. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Yet another embodiment is the use of the compositions and formulations described herein for treating pain, pruritus, or inflammation in a subject. Frequently, the pain, pruritus, or inflammation can be acute and can be due to a variety of causes such as allergies, insect bites, exposure to venom, poison ivy, atopic dermatitis, psoriasis, thermal burns, ionizing radiation, exposure to chemicals, trauma, surgery, nerve compression, oral or throat ulcers, bacterial infections, or viral infections. Alternatively, the pain, pruritus, or inflammation can be chronic and can be due to a variety of causes such as atopic dermatitis, psoriasis, viral infections, nerve compression, back pain, amputation, or trauma. In some situations, the pain, pruritus, or inflammation can be neuropathic and can be due to a variety of causes such as post herpetic neuralgia, back pain, nerve compression, viral infections, multiple sclerosis, Parkinson's disease, diabetes, trauma, amputation, or drug use.

In yet another embodiment, the compositions and formulations described herein can be administered topically to keratinized skin or mucous membranes in the eye, mouth, throat, esophagus, gastrointestinal tract, respiratory tract, or genitourinary tract.

In a generally applicable first aspect (i.e. independently combinable with any of the aspects or embodiments identified herein), a topical formulation is provided, comprising: a strontium moiety; and a polyacrylate crosspolymer-6 amphiphilic polymer.

In an embodiment of the first aspect, which is generally applicable (i.e., independently combinable with any of the aspects or embodiments identified herein), the topical formulation contains at least 5 wt. % elemental strontium, for example, from 5 wt. % to 14 wt. % or more elemental strontium, from 5 wt. % to 13 wt. % elemental strontium, from 5 wt. % to 12 wt. % elemental strontium, from 5 wt. % to 11 wt. % elemental strontium, from 5 wt. % to 10 wt. % elemental strontium, from 6 wt. % to 9 wt. % elemental strontium, from 7 wt. % to 9 wt. % elemental strontium, from 7 wt. % to 8 wt. % elemental strontium, from 8 wt. % to 12 wt. % elemental strontium.

In an embodiment of the first aspect, which is generally applicable (i.e., independently combinable with any of the aspects or embodiments identified herein), the topical formulation contains from 0.05 wt. % or less to 6.0 wt. % or more polyacrylate crosspolymer-6 amphiphilic polymer, from 0.1 wt. % to 5.0 wt. % polyacrylate crosspolymer-6 amphiphilic polymer, from 0.1 wt. % to 4.0 wt. % polyacrylate crosspolymer-6 amphiphilic polymer, from 0.1 wt. % to 3.0 wt. % polyacrylate crosspolymer-6 amphiphilic polymer, from 0.1 wt. % to 2.0 wt. % polyacrylate crosspolymer-6 amphiphilic polymer, from 0.1 wt. % to 1.5 wt. % polyacrylate crosspolymer-6 amphiphilic polymer, from 0.5 wt. % to 1.5 wt. % polyacrylate crosspolymer-6 amphiphilic polymer, from 0.1 wt. % to 0.9 wt. %, from 0.2 wt. % to 0.9 wt. %, from 0.3 wt. % to 0.9 wt. %, from 0.4 wt. % to 0.9 wt. %, from 0.5 wt. % to 0.9 wt. %, from 0.6 wt. % to 0.9 wt. %, from 0.7 wt. % to 0.9 wt. %, from 0.8 wt. % to 0.9 wt. %, from 0.6 wt. % to 0.9 wt. %, from 0.6 wt. % to 0.8 wt. %, from 0.6 wt. % to 0.7 wt. %, from 0.7 wt. % to 0.9 wt. %, from 0.7 wt. % to 0.8 wt. %, or from 0.1 wt. % to less than 1 wt. %.

In an embodiment of the first aspect, which is generally applicable (i.e., independently combinable with any of the aspects or embodiments identified herein), the topical formulation has a pH of less than 6, for example, a pH of less than 5, a pH of 2 to 6, a pH of 2 to 5, a pH of 2 to 4, a pH of 2 to 3, a pH of 3 to 5, a pH of 3 to 4, a pH of 4 to 6, or a pH of 4 to 5.

In an embodiment of the first aspect, which is generally applicable (i.e., independently combinable with any of the aspects or embodiments identified herein), the topical formulation has a viscosity of 50 centipoise or less to 2000000 centipoise or more, for example, a viscosity of 1000 centipoise to 100000 centipoise, a viscosity of 1000 centipoise to 90000 centipoise, a viscosity of 1000 centipoise to 80000 centipoise, a viscosity of 1000 centipoise to 70000 centipoise, a viscosity of 1000 centipoise to 60000 centipoise, a viscosity of 1000 centipoise to 50000 centipoise, a viscosity of 1000 centipoise to 40000 centipoise, a viscosity of 1000 centipoise to 30000 centipoise, a viscosity of 1000 centipoise to 20000 centipoise, a viscosity of 1000 centipoise to 10000 centipoise, a viscosity of 5000 centipoise to 100000 centipoise, a viscosity of 5000 centipoise to 90000 centipoise, a viscosity of 5000 centipoise to 80000 centipoise, a viscosity of 5000 centipoise to 70000 centipoise, a viscosity of 5000 centipoise to 60000 centipoise, a viscosity of 5000 centipoise to 50000 centipoise, a viscosity of 5000 centipoise to 40000 centipoise, a viscosity of 5000 centipoise to 30000 centipoise, a viscosity of 5000 centipoise to 25000 centipoise, a viscosity of 5000 centipoise to 20000 centipoise, a viscosity of 5000 centipoise to 15000 centipoise a viscosity of 5000 centipoise to 10000 centipoise, a viscosity of 10000 centipoise to 30000 centipoise a viscosity of 10000 centipoise to 25000 centipoise, a viscosity of 10000 centipoise to 20000 centipoise, a viscosity of 10000 centipoise to 15000 centipoise, a viscosity of 7500 centipoise to 30000 centipoise a viscosity of 7500 centipoise to 7500 centipoise, a viscosity of 7500 centipoise to 20000 centipoise, or a viscosity of 7500 centipoise to 15000 centipoise.

In an embodiment of the first aspect, which is generally applicable (i.e., independently combinable with any of the aspects or embodiments identified herein), the strontium moiety is a strontium salt selected from the group consisting of strontium nitrate, strontium chloride, strontium chloride hexahydrate, strontium sulfate, strontium carbonate, strontium hydroxide, strontium hydrosulfide, strontium oxide, strontium acetate, strontium mono-carboxylic acid, strontium di-carboxylic acid, strontium tri-carboxylic acid, strontium quatro-carboxylic acid, strontium amino carboxylic acid, strontium glutamate, strontium aspartate, strontium malonate, strontium maleate, strontium citrate, strontium threonate, strontium lactate, strontium pyruvate, strontium ascorbate, strontium alpha-ketoglutarate, and strontium succinate.

In an embodiment of the first aspect, which is generally applicable (i.e., independently combinable with any of the aspects or embodiments identified herein), the strontium moiety is strontium nitrate or strontium chloride.

In an embodiment of the first aspect, which is generally applicable (i.e., independently combinable with any of the aspects or embodiments identified herein), the topical formulation further comprises at least one active agent selected from the group consisting of analgesics, corticosteroids, sunscreen, vitamins, insect repellent, humectants, moisturizers, soothing agents, penetration enhancers, and combinations thereof.

In an embodiment of the first aspect, which is generally applicable (i.e., independently combinable with any of the aspects or embodiments identified herein), the topical formulation further comprises least one excipient selected from the group consisting of solvents, emulsifying agents, dispersants, thickeners, wetting agents, surfactants, foaming agents, defoaming agents, lubricants, evaporation reducers, preservatives, stabilizers, antioxidants, antimicrobials, reducing agents, fragrances, dyes, and combinations thereof.

In a generally applicable second aspect (i.e. independently combinable with any of the aspects or embodiments identified herein), a method is provided of manufacturing the topical formulation of the first aspect or any one of its embodiments, comprising: wetting the polyacrylate crosspolymer-6 polymer in an oil; dissolving the strontium moiety into an aqueous phase; and combining the wetted polyacrylate crosspolymer-6 polymer and the dissolved strontium moiety to create an emulsion.

In an embodiment of the second aspect, which is generally applicable (i.e., independently combinable with any of the aspects or embodiments identified herein), the method further comprises adding to the wetted polymer at least one member of the group consisting of a solvent, a thickener, a humectant, a surfactant, an emulsifying agent, or combinations thereof.

In a generally applicable third aspect (i.e. independently combinable with any of the aspects or embodiments identified herein), a method of treating pain or pruritus is provided, comprising: administering to a patient in need thereof a therapeutically effective amount of the topical formulation of the first aspect or any of its embodiments.

In an embodiment of the third aspect, which is generally applicable (i.e., independently combinable with any of the aspects or embodiments identified herein), the pain or pruritus is acute pain or acute pruritus due to allergies, insect bites, exposure to venom, poison ivy, atopic dermatitis, psoriasis, thermal burns, ionizing radiation, exposure to chemicals, trauma, surgery, nerve compression, oral or throat ulcers, bacterial infections, or viral infections.

In an embodiment of the third aspect, which is generally applicable (i.e., independently combinable with any of the aspects or embodiments identified herein), the pain or pruritus is chronic pain or chronic pruritus due to or associated with atopic dermatitis, psoriasis, a viral infection, nerve compression, back pain, an amputation, or trauma.

In an embodiment of the third aspect, which is generally applicable (i.e., independently combinable with any of the aspects or embodiments identified herein), the pain or pruritus is neuropathic pain or neuropathic pruritus due to or associated with post herpetic neuralgia, back pain, nerve compression, a viral infection, multiple sclerosis, Parkinson's disease, diabetes, trauma, amputation, or drug use.

In an embodiment of the third aspect, which is generally applicable (i.e., independently combinable with any of the aspects or embodiments identified herein), the topical formulation is topically administered to at least one member of the group consisting of keratinized skin, mucous membranes in the eye, mucous membranes in the mouth, mucous membranes in the throat, mucous membranes in the esophagus, mucous membranes in the gastrointestinal tract, mucous membranes in the respiratory tract, and mucous membranes in the genitourinary tract.

Any of the features of an embodiment of the first through third aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments. Any aspect or embodiment of a method can be performed using a composition of another aspect or embodiment, and any aspect or embodiment of a composition can be prepared by a method or utilized in a method of another aspect or embodiment

### DETAILED DESCRIPTION

The present disclosure consists of therapeutically-active compositions that combine strontium with a polymer that increase the overall therapeutic potency of the combination beyond the potency of any of the separate constituents. Specifically, the combinations described herein, as compared to strontium alone, increases the ability of topically-applied strontium to inhibit both acute sensory irritation (e.g., pruritus and pain), redness, swelling and inflammation (collectively defined for purposes of this description, “irritation”) and the chronic irritation that is characteristic of and contributes to the development and maintenance of painful or pruritic neuropathic conditions.

Accordingly, the present disclosure relates, in part, to compositions that include combinations of divalent strontium cations and a specialized polymer. The specialized polymers, when hydrated, form a three-dimensional structure that is mainly hydrophobic on the exterior and mainly hydrophilic on the interior.

In the description that follows, a number of terms are extensively utilized. Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When the terms “one,” “a,” or “an” are used in this disclosure, they mean “at least one” or “one or more,” unless otherwise indicated.

As used herein, the term “and/or” may mean “and,” it may mean “or,” it may mean “exclusive-or,” it may mean “one,” it may mean “some, but not all,” it may mean “neither,” and/or it may mean “both.”

As used herein, the term “strontium moiety” refers to either a strontium cation or a strontium salt. The term “elemental strontium” is conveniently employed in the context of the disclosure to refer to an amount or concentration of strontium cation (Sr⁺⁺) present in a formulation, on a weight basis. When a percentage of strontium or any other component of a formulation is referred to, the percentage is a percentage by weight, unless otherwise specified.

The phrase “high concentration” as used herein refers to an elemental strontium concentration of at least 5%.

As used herein, “treatment” means any manner in which the symptoms of a condition, disorder or disease are ameliorated or otherwise beneficially altered. Treatment also encompasses any pharmaceutical use of the compositions herein.

As used herein, “subject” refers to an animal, including, but not limited to, a mammal, for example, a primate (e.g., human). The terms “subject” and “patient” are used interchangeably herein. Both human and veterinary uses are contemplated.

### Nociception and Pain Transmission

Nociception involves the neural processes of encoding and processing stimuli that have the potential to damage tissue. Nociceptors are specialized nerves located throughout the body that detect mechanical, thermal or chemical changes. There are two classes of nociceptors, the first class is “A-delta” nerves, which respond to physical trauma by transmitting a pain sensation with a sharp, pricking quality. The second class is “Type C” nerves (TCN), which are chemical sensors that respond to irritants from our environment, such as microbes, temperature extremes, and ionizing radiation and transmit diffuse sensations of burning pain, stinging pain or itching (“irritation”). When excessively stimulated, TCN can also release neuropeptides (e.g., Substance P) that directly activate histamine-containing mast cells and attract and activate other immune system cells such as neutrophils that cause redness, swelling and even local tissue damage. After activation by a stimulus, nociceptors synapse near the spinal cord in the dorsal root ganglia (DRG) and release neurotransmitters that activate nerve pathways that relay signals to the brain. The brain interprets the signals as various types of pain or itch.

A. Acute, Chronic & Neuropathic Pain & Pruritus Occur Upon Nociceptor Activation

Exposure to stimuli activates nociceptors. Depending on the stimuli, both types of nociceptors may be activated or in many instances either the A-delta or TCN are preferentially activated. Since only the TCNs extend to the outermost portions of the body, such as the skin, mouth, nose, throat, eyes, etc. (herein referred to as “epithelium” or “epidermis”) and may be activated by virtually any process that changes the local biochemistry of the epidermis, TCNs are preferentially activated in response to most irritating stimuli. Upon activation of TCNs in the skin, the TCNs transmit a signal to the spinal cord and trigger neurotransmitter release in the DRG that activate nerves in the spinal cord that relay the pain and itch signals to the brain. Acute activation of TCNs that is caused by exposure to a chemical irritant, trauma or a sunburn typically causes painful or pruritic sensations that last only several days and is termed “nociceptive pain”. When the stimulus is prolonged or excessively severe as can occur after a viral disease like shingles or HIV, or the nerves are damaged by trauma to nerves from physical pressure, thermal burns, diabetes or extensive physical trauma to a limb, painful sensations or pruritus can continue for many years. Such chronic pain or pruritus caused by excessive nociceptor activation or damage is termed “neuropathic” and is among one of the most difficult conditions to treat. Even the best oral or topical drugs have only a very limited therapeutic benefit and many have substantial side effects that limit their use.

B. Nociceptive Signals are Typically Encoded as Precisely-Timed Changes of Intracellular Calcium Concentration that Travel as “Calcium Waves” within Nociceptors

No matter what causes nociceptor activation, the event is encoded into a universal code; a complex change in the intracellular calcium concentration that, in turn, is transmitted throughout the nociceptor. Calcium thus acts as a universal “second messenger” and information transmitted by a nociceptor, including the intensity and quality of pain or pruritus is converted into a language made up of rapidly changing calcium concentrations. Since nerves in general and nociceptors in particular transmit their calcium code typically within about 1/1000th of a second, the timing and spatial distribution of calcium must be exquisitely regulated to accurately transmit the encoded information. In virtually all nerves, including nociceptors, the intensity of the signal (e.g., the severity of pain or pruritus) is encoded as a change in frequency of neurotransmitters that are released into the synapse and activate post-synaptic nerves that relay the information ultimately to the brain. The higher the frequency, the more intense the perceived sensation. When a nociceptor is activated, the calcium signal is transmitted through multiple biochemical pathways, many of which operate in sequence such that the output of one pathway becomes the input of the next.

C. Nociceptive Signals and the Biochemical Pathways that Encode Signals have an Output that is Logarithmically Related to the Input

The many nociceptor pathways as well as the overall neurotransmitter release by a nociceptor are typically logarithmically related to the intensity of the stimulus. For example, if the irritant caused the nociceptor activation to increase its frequency of activation, also called depolarization, from 10 to 50 per second, the frequency of the resultant neurotransmitter release may only increase by a factor of 1.7 (Log 10=1.0; Log 50=1.7). This fact is particularly relevant to the inventors since it suggests that a relatively small amount of inhibition of a nociceptor's activation can cause a large reduction in the perceived severity of the painful or pruritic stimulus. Since there are many separate pathways in nociceptors that act in sequence to encode and transmit an irritant stimulus, inhibiting each of the sequential pathways at one or more of a pathway's steps has the potential to produce a very large cumulative reduction of the painful or pruritic sensation.

D. The Development and Maintenance of Neuropathic Pain or Pruritus Requires Excessive and Continuous Nociceptor Activation

In order for a neuropathic condition to develop, nociceptors must be continuously activated by a potent stimulus. The duration of the activation required may substantially vary depending on the specific nerve injury or stimulant. When such activation occurs, the peripheral nociceptors that innervate the skin and mucous membranes may become sensitized within hours and may continue to increase their sensitivity to irritants and may even be activated by stimuli that are normally not irritating. Infections such as HIV or Herpes viruses, or chronic colonization by bacteria such as Staphylococcus aureus that is present at excessive levels on the skin of atopic dermatitis patients, burn patients, patients suffering from ionizing radiation or traumatic damage to a nerve are especially potent nociceptor sensitizers. Release of multiple inflammatory mediators that accompany any trauma or inflammation are also important contributors to sensitization.

In order to establish a neuropathic state, sensory nerves in the DRG that receive sensory input from the TCN must also become sensitized. As for the peripheral TCN, the central neurons require sustained, high intensity activation for an extended period of time that may be as short as several weeks or much longer. The presence of inflammation, infectious agents, or trauma can accelerate the sensitized, neuropathic state. Due to neuronal “cross-talk,” it is common for an initially small painful portion of sensitized tissue, for example, as occurs in post-herpetic neuralgia, to expand to the adjacent tissue via nociceptors that were uninjured, including A-delta nociceptors. Sensitized neuropathic tissue may also generate painful stimuli in response to mechanical pressure, e.g. coughing or swallowing, or temperature changes, a condition known as allodynia.

The sensitized state in both the peripheral nociceptors and their central counterparts is a form of activity-dependent plasticity that is very similar to the neurons in the CNS that form memories. In the case of neuropathic pain or pruritus, the nociceptive response produces a “memory of pain or itching.” The molecules and pathways that produce the long-lasting neuronal sensitization are reasonably well defined, in particular, the activation of intracellular kinases. Of particular importance are protein kinase A and C (PKA and PKC, respectively), each of which exist in several different forms and the mitogen activated protein kinases (MAPK) that include the p38 MAPK, ERK1/2 MAPK and the JNK MAPK. These kinases are activated by a broad range of environmental “danger signals” and internal cytokines and growth factors exposures including ionizing radiation, reactive oxygen species (ROS) always accompany infection and trauma. When activated, these kinases are activated in multiple pathways and give rise to sequential cascades that result in regulation and activation of genes that regulate well over 100 different molecules that activate immune cells, produce inflammation and molecules that influence ion channels, and molecular sensors that cause the peripheral and central nociceptor sensitization that causes neuropathic pain and pruritus. Among these inflammation and immune-system activating genes, the most important is called Nuclear Factor, Immunoglobulin Light Chain Kappa, Enhancer of B Cells, abbreviated NF-Kappa B, called the “Master Gene Regulator of Inflammation.” Additionally, some of these kinases like PKC can directly sensitize and activate nociceptors that cause calcium influx and interfere with strontium's ability to alter the calcium dynamics that occur in neuropathic states.

There are many causes of neuropathies, some of which are very common. For example, common neuropathies include viral infection (e.g., HIV, the Herpes varicella zoster virus (VZV) that causes chicken pox and in later years, or secondary to immunosuppression, shingles and for many, post-herpetic neuralgia, an intensely painful condition that typically occurs in advanced age). Diabetes is the most common cause of the typical burning pain due to glucose-induced nerve damage, serious burns, severe trauma or amputation and a number of drugs, especially some that are used to treat HIV. While there are oral drugs available like gabapentin (e.g. NEURONTIN®) and pregabalin (e.g. LYRICA®) that can provide significant relief from neuropathic symptoms, they all have potentially significant side effects such as somnolence, dizziness and changes in mentation in more than 25% of patients. Since many neuropathic patients are in their 70s or 80s and already have health limitations, these side effects can be particularly problematic and potentially dangerous. This frequently leads to reduced compliance with the required dosing schedule and thus reduced patient benefit.

E. Stimuli that Oxidize Intracellular Glutathione Trigger Multiple Nociceptor-Activating Pathways

Of the many conditions that may cause nociceptor activation during the development of neuropathic conditions, the redox state of a nociceptor can produce some of the most potent acute and chronic nociceptor activating stimuli that exist. One of the most important regulatory signals that causes a cell to convert to a defensive state in which multiple inflammatory and cell protective immune activators are activated is the intracellular ratio of reduced glutathione (GSH) to oxidized glutathione (GSSG). Glutathione is the most plentiful intracellular thiol antioxidant, and is among the most important signal generators that trigger a cell to synthesize powerful inflammatory mediators and activate genes that, in turn, activate virtually every immune system inflammatory cell. The ratio of reduced glutathione, GSH, to the oxidized form, GSSH, is normally 9 to 1 or more. When cells are exposed to trauma, infection, inflammation or inflammatory mediators, ionizing radiation or general “cellular stress,” the amount of reduced glutathione plummets and directly triggers multiple cascades of gene activation that ultimately lead to the synthesis of well over 100 inflammatory mediators, pro-inflammatory cytokines (e.g., TNF-alpha, IL-1, IL-6 and many others), and cytokines that attract and activate inflammatory immune cells, all of which sensitize and activate nociceptors that transmit pain and pruritic signals, and in turn amplify these inflammatory cascades by neurogenic inflammatory pathways. Many of the most important cellular regulators of inflammation and immune defense are highly sensitive to a reduction in a cell's GSH concentration, and are directly activated by a low GSH/GSSG ratio indicating that a cell is in an oxidative redox state.

Perhaps the most important of these redox-sensitive regulatory pathways is NF-Kappa B. This molecule is responsible for directly or indirectly inducing the synthesis of the most important and powerful inflammation activators, including TNF-alpha and many of the inflammatory interleukins and chemokines that attract inflammatory cells that secrete mediators that directly activate nociceptors and thus increase their long-term sensitization and conversion to a neuropathic state.

Since NF-Kappa B acts as a “final common pathway” for activation of multiple inflammatory pathways, substances that reduce or block NF-Kappa B activation will have substantial and broad anti-inflammatory activity and will block many forms of immune system-mediated activation of inflammatory pathways. NF-Kappa B is also one of the many regulatory molecules that is directly activated by an oxidative intracellular environment—one in which the ratio of reduced glutathione (GSH) to oxidized glutathione (GSSG) is minimized. This oxidative environment directly activates NF-Kappa B that greatly increases the synthesis of nociceptor-activating mediators and cytokines.

Since both peripheral nociceptors with endings in the skin and central nociceptors in the DRG and spinal cord become sensitized upon continuous activation, activation of NF-Kappa B is an important and critical stimulator of neuropathic sensitization.

F. Activation of Toll-Like Receptors by Microbes Activate Gene Transcription by NF-Kappa B that Sensitizes Activate Nociceptors

Keratinocytes constitute about 90% of epidermal cells and have many receptors that can cause nociceptor activation. Among the most important are Toll-Like Receptors (TLRs), molecules that recognize conserved molecular structures of bacteria, fungi and viruses. Upon activation, TLRs trigger multiple inflammatory and nociceptor activating pathways, all of which lead to NF-Kappa B activation.

G. Activation of NF-Kappa B Produces Chemokines that Attract Inflammatory Cells

One of the most important consequences of NF-Kappa B is to stimulate the production of chemokines, including IL-8, that attract and activate neutrophils, a blood-borne white blood cell (WBC) that typically constitutes over 50% of all WBCs in the blood. Neutrophils are the first responders to any type of trauma, infection or inflammatory process and accumulate at the triggering site in massive quantities. Upon activation by IL-8 and other inflammatory mediators, neutrophils produce massive levels of powerful oxidants, reactive oxygen species (ROS; e.g., superoxide, hydrogen peroxide, nitric oxide and hypochlorous acid) that rapidly deplete GSH from cells, including nociceptors, thus promoting oxidative activation of NF-Kappa B and activation of many kinases, including Protein Kinase A, Protein Kinase C and Mitogen-Activated Protein Kinases that act to amplify virtually all inflammatory pathways that directly activate nociceptors.

Activation of these multiple independent inflammatory pathways and inflammatory cells results in intense activation of nociceptors that contributes to the development of neuropathic sensitization and neuropathic pain and pruritus.

Such activation of nociceptors also causes them to release Substance P that directly triggers mast cell activation and release of histamine, TNF-alpha, IL-1, IL-6, IL-8 and many more inflammatory substances that further activate nociceptors. Due to the simultaneous activation of multiple inflammatory and nociceptor-activating pathways, there is a net amplification of nociceptor activation that is known to directly lead to neuropathic pain and pruritus.

### Strontium's Effect on Pain Pathways

Strontium's unique therapeutic properties are due to its chemical resemblance to calcium, the most important and universal “second messenger” in nerves and in all other cells that regulate virtually all cellular functions. The calcium ion always has two positive charges and its ionic radius is 0.99 angstroms, about the size of a hydrogen atom. Of all the elements, strontium most closely resembles calcium, since it also only exists as a divalent positively-charged ion and has an ionic radius of 1.13 angstroms. For this reason, strontium typically binds to calcium-binding sites and mimics calcium's activity. Most often a strontium-induced response is less potent and may be as low as about 1/1000th as active as calcium, but for certain calcium-dependent activities, strontium has activity that is nearly the same as calcium or in the range of 1/10th to 1/30th as active as calcium. In other calcium-dependent activities, strontium can be more active than calcium. It is strontium's calcium-mimetic activity that enables strontium to produce its many and varied activities. Since calcium is critical for so many cellular functions, if it were strongly inhibited the effects would be toxic to a cell. In contrast, since strontium can typically substitute for calcium, albeit with lower activity, the activity of the calcium-dependent pathway will not be shut down. Instead, the pathway activity will be reduced, similar to turning down the volume control of a radio. Since strontium, in a metaphoric sense, only turns down the volume control of calcium-dependent pathways rather than shutting down such pathways, the chances of significant adverse reactions or toxicity is much reduced compared to a drug that completely blocks a pathway.

A. Strontium Alters the Dynamics and Spatial Distribution of Calcium Waves

When irritants from chemicals, disease, trauma or other exposures activate receptors on the surface of TCNs that encode the intensity of their response as rapid changes in intracellular calcium concentrations, these changes can occur in less than 1/1000th of a second and produce highly complex “waves” of changing calcium concentration that propagate through the nerve and trigger most, if not all, of the pathways that cause acute, chronic and neuropathic irritation. In addition to the frequency of calcium waves, alterations in the dynamics of calcium concentration change the duration, magnitude and the precise shape of the calcium waveform that alters the coexisting electrostatic field that is a critical regulator of TCN activity. These changes independently activate the release of multiple inflammatory mediators, including prostaglandins (e.g., PGE2), leukotrienes (e.g., LTB4, C4, D4 & E4) and reactive oxygen species (ROS) including superoxide, hydrogen peroxide, hydroxyl radicals, hypochlorous acid and peroxynitrite.

Strontium thus significantly alters the pain and itch sensations encoded within calcium waves present in painful and pruritic neuropathic conditions, and has the effect of distorting the signal and reducing its perceived intensity by the brain. Due to strontium binding to multiple calcium-dependent signaling pathways, strontium significantly alters calcium-encoded signals by multiple independent mechanisms. Some of the calcium-dependent kinases are known to be essential for the development of neuropathic conditions, since their inhibition in animal models can prevent and or reverse established neuropathic conditions.

Strontium is not able to bind effectively to the calcium binding proteins within the cytoplasmic interior of nociceptors that normally remove calcium within less than a millisecond after calcium enters the nociceptor, thus producing a transient increase in calcium concentration that contributes to the precisely-timed calcium waves. Strontium is also much less effectively pumped into and released from a nociceptor's primary calcium storage site, the endoplasmic reticulum (ER). When a nociceptor-activating signal is received, strontium inhibits the calcium-induced calcium release (CICR) pathway that amplifies the calcium signal, and strontium does not have the ability to regulate inositol triphosphate (IP3)-induced calcium release by acting to inhibit additional calcium release if the concentration of calcium in the cytoplasm is too high.

Once calcium enters a nociceptor during its activation and depolarization, it activates the release of a massive amount of calcium that is stored in the ER by the CICR pathway. This mechanism has the effect of greatly amplifying the amount of calcium that is available to form a wave and to regulate calcium-dependent pathways. Strontium is over a hundred-fold less active than calcium in its ability to induce CICR and thus significantly alters the calcium concentration changes that normally occur in response to irritants. When in the ER, strontium also binds much less avidly to the ER calcium binding proteins that act as buffers and sequester the free calcium until it is released by CICR or other similar mechanisms. As a result, strontium reaches a concentration of more than 150% greater than calcium and displaces calcium from performing its amplifying function during CICR. Strontium is also much less active then calcium in regulating a second important calcium amplifying mechanism triggered by IP3, a ubiquitous substance that also activates calcium release from the ER by an IP3-specific receptor. At low concentrations of calcium, IP3 acts as a potent stimulator of calcium release that acts to amplify the much smaller calcium influx during depolarization. When the calcium concentration is sufficiently elevated, calcium acts to inhibit further calcium release thus maintaining the calcium concentration within a limited concentration range. When strontium is present, it can mimic calcium in its ability to activate IP3-induced calcium release, but strontium is not able to inhibit excessive calcium release causing both calcium and strontium to reach higher concentrations over an extended time. Strontium's ability to completely inhibit calcium-induced release due to IP3 is particularly important, since IP3-induced calcium release is known to be responsible for generation of calcium waves. These types of strontium effects significantly change the calcium dynamic s and calcium waveforms associated with neuropathic conditions, and thus contribute to strontium's suppressive effects on pain and pruritus.

B. Strontium Inhibits Calcium-Dependent Neurotransmitter Release

While strontium also affects additional pathways that control the dynamics of calcium within nociceptors, there is one strontium-induced interference with calcium-dependent transmission of pain and itch-encoded calcium waves that is critically important for suppression of acute, chronic and neuropathic conditions. That is, the ability of strontium to bind and inactivate synaptotagmin-1, a molecule that is principally responsible for neurotransmitter release in the DRG and release of inflammatory neuropeptides, including substance P from the peripheral portion of a TCN in the skin. Substance P is known to be the most important inflammatory neuropeptide released from TCNs that activates virtually every inflammatory immune “white blood cell” (WBC), including mast cells that contain histamine and over 50 different inflammatory chemicals, including Tumor Necrosis Factor-alpha (TNF-alpha), Interleukin 1 alpha and beta (IL-1 alpha & beta) and IL-6. These three pro-inflammatory cytokines are believed to be the “first responders” that directly activate TCNs to cause pain and/or itching and are thought to be significant contributors to the development and maintenance of neuropathic conditions, as well as most skin conditions that are associated with inflammation, pain or itching.

Synaptotagmin-1 is a protein present on the surface of vesicles that contain and ultimately release neurotransmitters and anti-inflammatory neuropeptides like substance P from the pre-synaptic endings that bind to the post-synaptic neurons in the DRG and the peripheral TCN endings in the skin that relay the pain and itch-encoded signals to the brain. Normally, the frequency of the presynaptic neurotransmitter release from nociceptors are precisely matched so that the intensity, timing and other properties of the original pain or itch signal encoded in the calcium wave is accurately transmitted to the brain. The delay between the arrival of the calcium wave, neurotransmitter release and post-synaptic activation is usually about 1/1000th of a second and the amount released is related to the intensity of the original TCN signal. This type of neurotransmission is termed “synchronous release,” since the timing of the arrival of the calcium wave is tightly synchronized to the release of neurotransmitters that trigger post-synaptic activation of the DRG nerve. Without this precise coupling, the frequency encoded pain or itch signal becomes distorted.

When strontium substitutes for calcium, the amplitude of synchronous neurotransmitter release in response to TCN activation is typically reduced by more than 90%. Strontium has an additional signal distorting effect that significantly distorts the timing of neurotransmitter release called “asynchronous release.” In contrast to synchronous release that is tightly coupled to the stimulating signal, asynchronous release may extend to several hundred milliseconds. With strontium, the total amount of neurotransmitter that is released may be the same as with calcium, however the strength of the synchronous release that contains the encoded pain or itch intensity information is strongly reduced, and the critical timing information is essentially destroyed. This strontium mechanism not only reduces the perceived severity of a pain or itch signal, but it also suppresses the release of substance P at the proximal end of the TCN in the skin at the original site of TCN activation. Strontium's ability to inhibit the release of TNF-alpha, IL-alpha and IL-6 from keratinocytes is probably due to the same synaptotagmin-induced release mechanism since it is the secretory mechanism used by virtually every cell. Suppression of synchronous neurotransmitter release also has an important therapeutic benefit for neuropathic pain or pruritus treatment.

Accordingly, in one embodiment, it is therefore desirable to further alter the calcium dynamics of nociceptors by further suppressing calcium release or by interfering with critical calcium-dependent pathways that are partially inhibited by strontium.

C. Strontium Binds to a Calcium-Sensing Receptor (CaSR) on Nociceptors that Suppresses Nociceptor Activation

Most, if not all, cells have a recently-identified surface receptor (CaSR) that detects extracellular calcium concentrations. Strontium also binds and activates the CaSR receptor as efficiently as calcium, but triggers additional activities. In view of this, a simple strontium salt was commercially developed, strontium ranelate, which is an orally administered prescription drug for osteoporosis treatment in over 70 countries. Due to strontium's unique ability to mimic calcium's ability to activate the CaSR and, additionally, to activate additional pathways linked to the CaSR, strontium ranelate is the only known osteoporosis drug that has two independent osteoporosis therapeutic mechanisms—strontium inhibits bone loss by inhibiting bone-resorbing osteoclasts, and simultaneously stimulates osteoblasts that produce new bone.

Nociceptors also have a CaSR that inhibits nociceptor activation when the extracellular concentration of calcium is raised above normal, or if a similar concentration of strontium is administered. This mechanism contributes to the ability of strontium to rapidly inhibit TCN activation by, for example, highly acidic chemical peels such as 70% glycolic acid, pH 0.6, that cause burning pain within seconds after application. When strontium is mixed with the acid, burning pain and stinging is suppressed by 80% or more so that any remaining sensory irritation is not bothersome.

Activation of the CaSR also causes activation of several pathways that are known to increase both acute, chronic and neuropathic pain and pruritus and inflammation. Since in real world use, strontium typically inhibits pain and pruritus, it is likely that the pain and itch enhancing effect caused by activation of the CaSR by strontium is, in effect, negated by other strontium anti-irritant mechanisms. None the less, even a low level, “subclinical’ pain and itch-enhancing effect reduces the ability of strontium to effectively treat, prevent or reverse neuropathic conditions for which any excess TCN activation is known to promote the neuropathic condition.

Of particular concern is strontium's reported ability to bind to the CaSR and rapidly activate two of the MAPK molecules, p38 and ERK1/2, that are known to be among the primary contributors to peripheral and central nociceptor sensitization. Strontium binding to the CaSR is also reported to activate an important enzyme, Phospholipase C, that produces two important regulatory molecules, the aforementioned IP3, and diacylglycerol (DAG), both of which contribute to nociceptor activation and sensitization and inflammation. IP3 is one of the most important and potent calcium releasing molecules that directly trigger calcium release from ER stores. Many of the pain and itch producing chemicals that are produced during inflammation, infection or trauma use the IP3 pathway to activate nociceptors and produce the calcium waves that transmit pain and itch sensations. DAG is the principle activator of Protein Kinase C, a family of molecules that directly activates nociceptors and many of the pathways that produce pain and itch and inflammatory mediators. PKC is also known to be an important nociceptor sensitizer, since PKC inhibition can prevent or reverse neuropathic pain in animal models. PKC also activates NF-Kappa B, one of the most important stimulators of molecules that trigger pain, pruritus and inflammation and are thought to be able to directly cause neuropathic sensitization. It should be emphasized that the recognition that strontium produces its osteoporosis therapeutic benefits by binding to the CaSR is very recent and additional strontium-sensitive pathways will likely be identified. The fact that human nociceptors have the CaSR that regulates nociceptor activation suggests that CaSR activation by topically-applied strontium may be working at a reduced level due to strontium's ability to inhibit important pain and itch pathways while simultaneously activating pathways via the CaSR that are known to trigger pain and itch pathways. Most importantly, since activation of these CaSR pathways is known to contribute to the development of neuropathic conditions, strontium's therapeutic potential may be substantially compromised.

Accordingly, in one embodiment, it is therefore desirable to create strontium-based formulations that have molecular components that specifically inhibit the CaSR pathways known to enhance neuropathic pain, pruritus and inflammation.

### Compositions

One objective of the present disclosure to create effective topical drug formulations that are sufficiently safe to be used as needed and without fear of medically significant side effects, and that can effectively treat pain and pruritus. After strontium was first commercialized, it became clear that while strontium was safe and effective in many commercial applications, it suffered from a number of deficiencies that ultimately limited its potential therapeutic utility. For example, strontium salt at a concentration of 2-6% (equivalent to about 0.5-2% elemental strontium) in a formulation frequently caused transient stinging and redness/swelling if the treated skin was broken or had a damaged ‘barrier’ due to trauma, chemical exposure, infection or disease. Patients with ‘diaper rash’, both infants and people who are incontinent frequently experienced intense pain described as stinging for 5-10 seconds when using a 4 or 6% strontium salt (equivalent to about 1-2% elemental strontium) formulation. While not harmful, it was not tolerable for many infants. Similarly, strontium at higher concentrations was not applied to thermal burns, cuts or skin that had been highly excoriated due to scratching. Even on intact skin, some users would experience stinging when using a high strontium salt concentration gel or spray formulation, especially if the skin was excessively or chronically dry as occurs in atopic dermatitis. Without wishing to be bound by any one theory, it is believed that high osmolarity formulations activate specific osmotic sensors present on nociceptors, keratinocytes and immune or inflammatory cells that can activate nociceptors and trigger both sensory irritation like stinging and burring and erythema and swelling. An example of this is the “salt in the wound” effect that causes stinging and burning if a concentrated solution of a simple salt is poured into wound. In addition to causing discomfort, high osmolarity solutions can directly activate inflammatory cells and cause them to release chemicals that cause nociceptor activation. It is therefore desirable to minimize the potential for osmotic-induced nociceptor activation and inflammation.

The compositions and formulas of the present disclosure are designed around three general design & therapeutic principles: (1) an “extended release” strontium based formulation, (2) a high strontium concentration formula that does not trigger the “salt in the wound” effect, and (3) a shelf-stable high concentration strontium formulation.

A. Components

Previous attempts to develop emulsion-based lotions or creams were limited by the inherent emulsion destabilizing effect of electrolytes like strontium and its counter ions that disrupt the very electrostatic forces that create emulsions. At most, emulsions could achieve 4-5% strontium salt (e.g. 2% elemental strontium), however, the resulting formulation was so thick and sticky, it was difficult to apply to the skin. This problem occurred even when using thickeners that were designed for high salt formulations.

In one embodiment, the compositions of the present disclosure are high concentrations of strontium salts in a stable emulsion. The concentration of elemental strontium can reach at least about 12% or higher in compositions of the present disclosure. These concentrations are achievable through the use of, for example, specially designed polymers described herein. Exemplary strontium salts and polymers are discussed herein and below.

1. Strontium

Strontium is present as a divalent cation. Strontium is designated by its commonly used atomic symbol, ‘Sr’ and is depicted below.

Strontium mimics the ability of calcium to pass through voltage dependent calcium channels. As such, it may compete with Ca++ for binding to some receptors. Calcium is thought to play a role in the pain process by regulating the release of neurotransmitters, and thus strontium's analgesic effect may be in preventing calcium's binding to nerve cells.

Strontium is available as an inorganic or organic salt which is water soluble at room temperature in the range of 1 to 100 g/l. Inorganic salts include, for example, strontium chloride, strontium sulfate, strontium carbonate, strontium nitrate, strontium hydroxide, strontium hydrosulfide, strontium oxide, strontium acetate, etc. Organic salts include, for example, negatively charged organic acid such as a mono-, di-, tri- or quatro-carboxylic acid, or an amino carboxylic acid that may have a linear or branched carbon chain of from 2 to 30 carbon atoms and one or more amino groups attached thereto. The amino carboxylic acid may be a natural or synthetic amino acid. Examples of organic strontium salts include, for example strontium glutamate, strontium aspartate, strontium malonate, strontium maleate, strontium citrate, strontium threonate, strontium lactate, strontium pyruvate, strontium ascorbate, strontium alpha-ketoglutarate or strontium succinate. Other examples of strontium salts, and methods for preparation thereof, can be found, for example, in US Patent Application Pub. No. 2010/0048697.

The percentage of elemental strontium in a strontium salt can be determined by dividing the molecular weight of strontium by the molecular weight of the salt and multiplying by 100. For example, if using strontium nitrate, the molecular weight of strontium is 87.62 grams/mole (g/mol) and the molecular weight of strontium nitrate is 211.63. Accordingly, the percentage of strontium by weight in strontium nitrate is 41.4%. A formulation containing 20% strontium nitrate would yield 8.3% elemental strontium.

2. Polymers

The polymers disclosed herein are capable of forming stable emulsions containing high concentrations of strontium cations and/or strontium salts without becoming too thick or sticky.

In one embodiment, the compositions of the present disclosure include a strontium cation or strontium salt and a polymer capable of ionic association with the strontium cation or strontium salt, in which case the combination forms a matrix. Such matrix formation enhances the bioavailability of the strontium cations and therefore prolongs the therapeutic effect of such formulations. The matrix also reduces the amount of strontium that initially is absorbed by the skin, thus reducing osmotic shock.

In another embodiment, specialized polymers can be used to create shelf stable high strontium salt emulsions. In another embodiment, the specialized polymer is capable of stabilizing emulsion formulations containing at least 5% of elemental strontium. In another embodiment, the specialized polymer is capable of stabilizing emulsion formulations containing at least 6% of elemental strontium. In another embodiment, the specialized polymer is capable of stabilizing emulsion formulations containing at least 7% of elemental strontium. In another embodiment, the specialized polymer is capable of stabilizing emulsion formulations containing at least 8% of elemental strontium. In another embodiment, the specialized polymer is capable of stabilizing emulsion formulations containing at least 9% of elemental strontium. In yet another embodiment, the specialized polymer is capable of stabilizing emulsion formulations containing at least 10% of elemental strontium. In another embodiment, the specialized polymer is capable of stabilizing emulsion formulations containing at least 11% of elemental strontium. In another embodiment, the specialized polymer is capable of stabilizing emulsion formulations containing at least 12% of elemental strontium. In another embodiment, the specialized polymer is capable of stabilizing emulsion formulations containing at least 13% of elemental strontium. In another embodiment, the specialized polymer is capable of stabilizing emulsion formulations containing at least 14% of elemental strontium. In yet another embodiment, the specialized polymer is capable of stabilizing emulsion formulations containing at least 15% of elemental strontium.

In one embodiment, the specialized polymer is amphiphilic, meaning that it contains at least a hydrophilic portion and a hydrophobic portion.

In one embodiment, the amphiphilic polymer can be defined by a hydrophilic-lipophilic balance (HLB), which is a measure of the degree to which it is hydrophilic or lipophilic, determined by calculating values for the different regions of the polymer. A convenient calculation of HLB is equal to 20×Mₕ/M, where Mₕ is the molecular mass of the hydrophilic portion of the hydrophilic portion of the molecule, and M is the molecular mass of the whole molecule, giving a result on a scale of 0-20. In one embodiment, the amphiphilic polymer has a HLB of from about 1 to about 19, or about 2 to about 18, or about 3 to about 17, or about 4 to about 16, or about 5 to about 15, or about 6 to about 14, or about 7 to about 13, or about 8 to about 12, or about 9 to about 11, or about 10.

In certain embodiments, the amphiphilic polymer is equally hydrophobic and hydrophilic. In another embodiment, the amphiphilic polymer is greater than 50% hydrophobic, on the basis of a molecular mass of the hydrophobic portions of the polymer to total molecular mass of the polymer. In another embodiment, the amphiphilic polymer is greater than 60% hydrophobic. In another embodiment, the amphiphilic polymer is greater than 70% hydrophobic. In another embodiment, the amphiphilic polymer is greater than 80% hydrophobic. In another embodiment, the amphiphilic polymer is greater than 90% hydrophobic. In another embodiment, the amphiphilic polymer is 5% to 95% hydrophobic. In another embodiment, the amphiphilic polymer is 10% to 90% hydrophobic. In another embodiment, the amphiphilic polymer is 15% to 85% hydrophobic. In another embodiment, the amphiphilic polymer is 20% to 80% hydrophobic. In another embodiment, the amphiphilic polymer is 25% to 75% hydrophobic. In another embodiment, the amphiphilic polymer is 30% to 70% hydrophobic. In another embodiment, the amphiphilic polymer is 35% to 65% hydrophobic. In another embodiment, the amphiphilic polymer is 40% to 60% hydrophobic. In another embodiment, the amphiphilic polymer is 45% to 55% hydrophobic. In another embodiment, the amphiphilic polymer is about 50% hydrophobic. In another embodiment, the amphiphilic polymer is 50% to 95% hydrophobic. In another embodiment, the amphiphilic polymer is 50% to 90% hydrophobic. In another embodiment, the amphiphilic polymer is 50% to 85% hydrophobic. In another embodiment, the amphiphilic polymer is 50% to 80% hydrophobic. In another embodiment, the amphiphilic polymer is 50% to 75% hydrophobic. In another embodiment, the amphiphilic polymer is 50% to 70% hydrophobic. In another embodiment, the amphiphilic polymer is 50% to 65% hydrophobic. In another embodiment, the amphiphilic polymer is 50% to 60% hydrophobic. In another embodiment, the amphiphilic polymer is 50% to 55% hydrophobic.

In another embodiment, the amphiphilic polymer has a hydrophilic region. In another embodiment, the hydrophilic region is up to 5% of the total amphiphilic polymer, on the basis of a molecular mass of the hydrophilic portions of the polymer to total molecular mass of the polymer. In another embodiment, the hydrophilic region is up to 10% of the total amphiphilic polymer. In another embodiment, the hydrophilic region is up to 15% of the total amphiphilic polymer. In another embodiment, the hydrophilic region is up to 20% of the total amphiphilic polymer. In another embodiment, the hydrophilic region is up to 25% of the total amphiphilic polymer. In another embodiment, the hydrophilic region is up to 30% of the total amphiphilic polymer. In another embodiment, the hydrophilic region is up to 35% of the total amphiphilic polymer. In another embodiment, the hydrophilic region is up to 40% of the total amphiphilic polymer. In another embodiment, the hydrophilic region is up to 45% of the total amphiphilic polymer.

In another embodiment, the amphiphilic polymer is 5% to 95% hydrophilic, on the basis of a molecular mass of the hydrophilic portions of the polymer to total molecular mass of the polymer. In another embodiment, the amphiphilic polymer is 10% to 90% hydrophilic. In another embodiment, the amphiphilic polymer is 15% to 85% hydrophilic. In another embodiment, the amphiphilic polymer is 20% to 80% hydrophilic. In another embodiment, the amphiphilic polymer is 25% to 75% hydrophilic. In another embodiment, the amphiphilic polymer is 30% to 70% hydrophilic. In another embodiment, the amphiphilic polymer is 35% to 65% hydrophilic. In another embodiment, the amphiphilic polymer is 40% to 60% hydrophilic. In another embodiment, the amphiphilic polymer is 45% to 55% hydrophilic. In another embodiment, the amphiphilic polymer is about 50% hydrophilic. In another embodiment, the amphiphilic polymer is 5% to 50% hydrophilic. In another embodiment, the amphiphilic polymer is 5% to 45% hydrophilic. In another embodiment, the amphiphilic polymer is 5% to 40% hydrophilic. In another embodiment, the amphiphilic polymer is 5% to 35% hydrophilic. In another embodiment, the amphiphilic polymer is 5% to 30% hydrophilic. In another embodiment, the amphiphilic polymer is 5% to 25% hydrophilic. In another embodiment, the amphiphilic polymer is 5% to 20% hydrophilic. In another embodiment, the amphiphilic polymer is 5% to 15% hydrophilic. In another embodiment, the amphiphilic polymer is 5% to 10% hydrophilic.

In one embodiment, the polymer may form three dimensional (3D) structures that are roughly spherical or like a flattened sphere in shape. In one embodiment, the 3D structures can have an exterior portion and an interior portion, each having hydrophilic or hydrophobic properties. In on embodiment, the 3D structures can have an exterior that is mainly hydrophilic and the interior that is mainly hydrophobic. In another embodiment, the 3D structures can have an exterior that is mainly hydrophobic and an interior that is mainly hydrophilic. In another embodiment, the interior is at least 50% hydrophilic. In another embodiment, the interior is at least 60% hydrophilic. In another embodiment, the interior is at least 60% hydrophilic. In another embodiment, the interior is at least 70% hydrophilic. In another embodiment, the interior is at least 80% hydrophilic. In another embodiment, the interior is at least 90% hydrophilic. In another embodiment, the interior is at least 100% hydrophilic.

Without wishing to be bound by any one theory, it is believed that the hydrophobic/hydrophilic ratio as well as the location of the hydrophilic region contributes to the size of the 3D structure of the amphiphilic polymer. In another embodiment, the size of the 3D structures is in the micrometer range. In one embodiment, the diameter of the 3D structure is less than 50 micrometers. In another embodiment, the diameter is less than 40 micrometers. In another embodiment, the diameter is less than 30 micrometers. In another embodiment, the diameter is less than 20 micrometers. In another embodiment, the diameter is less than 10 micrometers. In another embodiment, the diameter is less than 5 micrometers. In another embodiment, the diameter is less than 4 micrometers. In another embodiment, the diameter is less than 3 micrometers. In another embodiment, the diameter is less than 2 micrometers.

In one embodiment, the specialized polymer has an acrylate backbone with one or more side chains attached to the carboxyl groups. Non-limiting examples of side chains include alkyl, cycloalkyl, alkenyl, alkynylamino, ester, ether, ammonium sulfonate, dimethylammonium, polyethylene glycol (PEG), or PEG/alkyl mix. The side chains may be either substituted or unsubstituted. In another embodiment, the polymer is 2-propenoic acid, 2-methyl-, dodecyl ester, polymer with ammonium 2-methyl-2-[(1-oxo-2-propen-1-yl)amino]-1-propanesulfonate(1:1), N,N-dimethyl-2-propenamide and α-(2-methyl-1-oxo-2-propen-1-yl)-ω-(dodecyloxy)poly(oxy-1,2-ethanediyl). The INCI name is polyacrylate crosspolymer-6 (herein referred to as “PC-6”). PC-6 is commercially available as SepiMAX™ ZEN (SEPPIC, Puteaux Cedex, France). PC-6 uses both electrostatic repulsion and hydrophobic interactions to stabilize high salt formulations. It is believed that in low salt conditions the electrostatic repulsion is the main stabilizing force whereas in high salt conditions, the hydrophobic interactions are the main stabilizing force.

PC-6 is mostly hydrophobic (e.g. 70-80%) and has a small (e.g. 20%) hydrophilic tail. When wetted or hydrated, PC-6 forms a roughly spherical 3D shape that is about 1-4 micrometers in diameter and has a hydrophobic exterior and hydrophilic interior.

PC-6 has a Number Average Molecular Weight (Mₙ)>10,000 Da.

In one embodiment, the amount of amphiphilic polymer (e.g., PC-6) can be from about 0.05% to about 5% of the total weight of the composition or formulation. In another embodiment, the amount of amphiphilic polymer (e.g., PC-6) can be from about 0.05% to about 3%. In another embodiment, the amount of amphiphilic polymer (e.g., PC-6) can be from about 0.1% to about 2%. In another embodiment, the amount of amphiphilic polymer (e.g., PC-6) can be from about 0.1% to about 0.1% to about 1%.

In another embodiment, the specialized polymer is polyvinylpyrrolidone (poly-[1-(2-oxo-1-pyrrolidinyl)-ethylene]), also known as PVP, polyvidone, povidon(e), povidonum and poly(l-vinyl-2-pyrrolidone). This compound will be referred to herein as PVP. PVP is a linear, amphiphilic, non-ionic, physiologically inert polymer made from N-vinylpyrrolidone, and has a linear formula of (C₆H₉NO)ₙ and a molecular weight range of about 2,500 to 2,500,000. Molecular weights as referred to herein are number average molecular weights, M_{w}, unless otherwise designated. PVP is highly water-soluble, and has been used extensively in the formulation of pharmaceutical systems, for example as a binder in the production of granules and tablets, as well as a polymer coating for granules and tablets, as a solubilizer for oral and parenteral formulations and as a viscosity-modifying agent in a variety of topical formulations. PVP can also serve as a bioavailability enhancer, taste masking agent, lyophilizing agent, suspension stabilizer, adhesive and drug stabilizer.

PVP is commercially available from many sources, including BASF (Florham Park, N.J.), Sigma-Aldrich (St. Louis Mo.), Cole-Parmer (Vernon Hills, Ill.) and SPI Supplies (West Chester, Pa.). The BASF products for use in the pharmaceutical industry are referred to as the Kollidon® grades, and include Kollidon®12 (MW 2,000-3,000), Kollidon® 12 PF (MW 2,000-3,000), Kollidon® 17 PF (MW 7,000-11,000), Kollidon® 25 (MW 28,000-34,000), Kollidon® 30 (MW 44,000-54,000), and Kollidon® 90 F (MW 1,000,000-1,500,000).

PVP solution viscosity does not change appreciably over a wide pH range. PVP is freely soluble in many organic solvents, including alcohols, some chlorinated compounds such as chloroform, methylene chloride and ethylene dichloride, nitroparaffins, and amines. Dilute solutions of PVP in hydrocarbons may be prepared by the use of a cosolvent, e.g., butanol, N-methyl-2-pyrrolidone. PVP shows a high degree of compatibility, both in solution and film form, with most inorganic salt solutions and with many natural and synthetic resins. PVP forms molecular adducts with many other substances. This can result in a solubilizing action in some cases or in precipitation in others.

Other suitable amphiphilic polymers for us in the compositions and methods described herein are poly(ethylene oxide) amphiphiles such as distearoylphohphatidylethanolamine-poly(ethylene glycol), and block copolymers including poly(ethylene oxide)-block-poly(ethylethylene), poly(ethylene oxide)-block-poly(caprolactone), poly(ethylene oxide)-block-polystyrene, poly(L-amino acids), poly(esters) and pluronics, also known as poloxamers, which are triblock copolymers based on poly(ethylene oxide)-block-poly(propylene oxide)-block-poly(ethylene oxide) which are typically expressed as PEO_{m/2}-b-PPOₙ-b-PEO_{m/2}. Many such polymers are well known in the art, and any polymer containing both a hydrophobic region and a hydrophilic region is suitable for use in the compositions and methods described herein.

In one embodiment, the number average molecular weight Mₙ of the amphiphilic polymer is from about 1,000 or less to about 100,000 or more. In another embodiment, the molecular weight range is from about 5,000 to about 50,000. In yet another embodiment, the molecular weight range is from about 10,000 to about 30,000.

3. Optional Ingredients

The strontium and amphiphilic polymers disclosed herein may optionally include an active agent such as those that provide therapeutic or protective properties. Non-limiting examples include analgesics, corticosteroids, sunscreen, vitamins, insect repellent, humectants, moisturizers, soothing agents, and penetration enhancers.

The strontium and amphiphilic polymers may include excipient ingredients that assist with formulation preparation or/use such as (but not limited to) solvents, emulsifying agents, dispersants, thickeners, wetting agents, surfactants, foaming agents, defoaming agents, lubricants, and evaporation reducers.

The strontium and amphiphilic polymers may include excipient ingredients that increase the shelf-life of the final product such as preservatives, stabilizers, antioxidants, antimicrobials, and reducing agents.

The strontium and amphiphilic polymers may include excipient ingredients that increase consumer appeal such as (but not limited to) fragrance and color.

The strontium and amphiphilic polymers disclosed herein may optionally include additional excipient ingredients that are known in the art.

B. Extended Release

The effects of strontium on blocking nociception are understood to be temporary. Though not wishing to be bound by any particular theory of operation, relief from the pain or itch lasts until the strontium is cleared from the nociceptor, which generally occurs within hours after exposure. To maintain relief, the strontium must be re-administered on a regular or periodic basis. Accordingly, the present inventors have determined that it would be beneficial to have a strontium compound that would provide longer lasting relief.

The synergistic interaction between strontium and PC-6 also allows for strontium to be released over a longer period of time. Without wishing to be bound by any one theory, it is believed that the availability of strontium occurs over three phases based on strontium's interaction with the PC-6 polymer (more information on the interaction between strontium and PC-6 can be found in the “Formulations and Manufacturing Methods” section below). The first phase is mainly unbound strontium cations. The second phase is mainly strontium cations forming a matrix by binding to the exterior of the 3D polymer spheres (i.e. one strontium cation can bind to two spheres). The third phase is mainly strontium cations located inside the 3D polymer spheres. Accordingly, it is believed that when the user applies the strontium emulsion the skin initially absorbs the unbound strontium. Over time, the breakdown of the 3D polymer spheres/strontium matrix releases the bound strontium, providing a second source of strontium for the skin to absorb. Last, the breakdown of the 3D spheres releases the strontium inside the spheres, providing a third source of strontium for the skin to absorb. This multiphase absorption provides longer lasting beneficial affects compared to single phase absorption.

C. Osmotic Shock

Strontium's anti-irritant activity is due to the divalent strontium ion. Pure strontium is highly reactive with oxygen and water. Accordingly, formulations containing strontium use strontium salts as the source of strontium. Due to its dual positive charges, two anionic counterions are required to balance the electrostatic charge and thereby create a strontium salt. With most commercially available strontium salts, the negatively-charged counterions, such as nitrate (NO3−) or chloride (Cl−) contribute to the ionic strength and osmolarity of the formulation, but not to the overall anti-irritant benefits. Furthermore, clinical studies have shown that higher strontium concentrations produce increased clinical benefits. Consequently, it is medically and commercially advantageous to create commercially acceptable and stable formulations with high strontium concentrations.

While high concentration strontium formulations would be clinically beneficial, they may also cause osmotic shock resulting in tissue damage and pain, especially in non-keratinized epithelium such as mucous membranes or in keratinized epithelium that has reduced barrier capabilities due to physical trauma, infection, or inflammation. Such hyperosmotic-induced damage is popularly known, as noted above, as the “salt in the wound effect” and it occurs when osmotic forces cause water to flow out of the cells and tissues into the hyperosmotic formulations. It is also believed that application of hyperosmotic formulations can directly activate certain molecules that act as osmolarity sensors and, when activated, activate pain sensing nerves and immune and non-immune cells that can produce inflammation and cellular damage. This recent understanding has potentially critical importance for the goal of preventing the development of chronic or neuropathic pain.

The potential importance of this observation has critical importance for the treatment of or the prevention of neuropathic pain development since chronic nociceptor activation is known to be required for painful neuropathic conditions to develop. The recent discovery that there are multiple ion channels and related hyperosmotic molecular sensors that trigger nociceptor activation upon exposure to hyperosmotic topical formulations suggests that their chronic use may predispose the development of neuropathic pain conditions if there is coexisting chronic or severe damage to nociceptors. In this scenario, long-term application of a hyperosmotic formulation to skin, and especially to delicate mucous membranes of, for example, the vaginal or cervical mucosa my cause low level, but long-term activation of nociceptors, thus contributing to their sensitization. It is believed that progression of from an acute, transient pain state to a chronic, long-lasting, ‘neuropathic state’ is due to continued excessive nociceptor activation that results in increased expression of genes that reduce the magnitude of an irritant stimuli, also called the irritant or nociceptor activation ‘threshold’ and thus cause increased nociceptor activation and an increased perception of pain and/or pruritus. Additionally, these genes can also increase the synthesis of inflammation-producing molecules that further irritate the nociceptors, thus producing what is commonly termed ‘a vicious spiral’ of increasing sensory irritation and inflammation.

In addition to causing painful or pruritic sensations and inflammation, even low-level, but chronic exposure to nociceptor-activating irritants can predispose to infection by a multitude of pathogenic microbes of which Herpes simplex viruses 1 and 2 (HSV) and the Human Immunodeficiency Virus (HIV) cause the greatest threat to public health. While a detailed explanation of the many and varied reasons for why nociceptor activation and coexisting inflammation facilitates infection by HSV and HIV is not discussed in detail herein, in essence, the release by Type C Nociceptors of inflammatory neuropeptides like substance P is known to damage the anatomical ‘barriers’ of both keratinized skin and mucosal membranes that block viral infection. The resultant inflammation is also known to activate inflammatory immune cells that, ironically, contribute to the ability of both HSV and HIV to cause acute infection and in the case of HSV, reactivation of an existing latent infection.

Application of hyperosmotic topical formulations of, for example, lubricants or microbicides, to the mucous membranes of male or female genitals or to the vaginal, cervical or anal tissues may greatly increase the possibility of transferring one of these viruses or other pathogenic microbes that cause sexually-transmitted diseases from an infected person to an otherwise healthy person. It is therefore be advantageous to create strontium-containing formulations with high strontium concentrations that are designed to minimize osmotic shock. One object of the present disclosure is to provide a high concentration strontium-containing formula having a minimal osmotic activity.

Another unexpected benefit of the synergistic combination of PC-6 and strontium in high concentration formulations is the reduction of osmotic shock. As alluded to in the previous section, “Extended Release,” and explained in more detail the “Formulations and Manufacturing Methods” section below, a majority of the strontium ions are bound to the PC-6 polymer, either internally or externally. The binding of the strontium ions can effectively reduce the available strontium ions when the compound is initially applied. The reduced strontium ions can result in a reduced risk of osmotic shock since the epidermis is exposed to a lower dose of salt.

D. Stability

Many formulations such as lotions, creams and hydrogels rely on a delicate balance of factors that produce stable emulsions or hydrogels. Formulations with high ionic strengths commonly prevent stable emulsion formation. Inventor's previous emulsions in which more than about 6-7% strontium nitrate or strontium chloride hexahydrate, (equivalent to about 2% elemental strontium) are incorporated were unstable and separated. Similarly, hydrogels containing more than about 12% to 13% (equivalent to about 4% elemental strontium) of these salts were also unstable. Since two thirds of most strontium salts represent ions that act to destabilize formulations, it was unexpectedly discovered that stable high concentration elemental strontium emulsions could be formulated using PC-6.

Another unexpected benefit of the synergistic combination of PC-6 and strontium is the ability to form stable emulsions containing high concentrations of elemental strontium. The unique interaction between the strontium cation and the PC-6 polymer allows for stable emulsions. Without wishing to be bound by any theory, it is believed that the strontium cations interact with the PC-6 polymer in at least two key ways. In the first way, the strontium cations bind to the outside of the 3D spheres formed by the PC-6 polymers. Since strontium is a divalent cation, it can bind to two different spheres, thus creating a matrix. In the second way, the strontium binds to or is trapped within the interior of the 3D spheres. Binding of the strontium reduces the amount of free strontium cations that can interfere with emulsion formation. Accordingly, through the use of PC-6, it is possible to create high concentration elemental strontium emulsions that are stable.

In one embodiment, the concentration of elemental strontium is at least 5%. In another embodiment, the concentration of elemental strontium is at least 6%. In another embodiment, the concentration of elemental strontium is at least 7%. In another embodiment, the concentration of elemental strontium is at least 8%. In another embodiment, the concentration of elemental strontium is at least 9%. In another embodiment, the concentration of elemental strontium is at least 10%. In another embodiment, the concentration of elemental strontium is at least 11%. In another embodiment, the concentration of elemental strontium is at least 12%. In another embodiment, the concentration of elemental strontium is at least 13%. In another embodiment, the concentration of elemental strontium is at least 14%. In another embodiment, the concentration of elemental strontium is at least 15%. In another embodiment, the concentration of elemental strontium is at least 16%.

### Formulations and Manufacturing Methods

The strontium compounds disclosed herein can be manufactured using standard formulation equipment. In one embodiment, the strontium compounds can be in the form of emulsions such as creams or lotions. In another embodiment, the compounds are ointments or pastes. In yet another embodiment, the compounds are liquid based such as gels or those used in sprays. The final form will dictate the formulation ingredients used as well as the manufacturing techniques. In one embodiment, the strontium is formulated as an emulsion. In another embodiment, the strontium is formulated as a lotion. In another embodiment, the strontium is formulated as a cream.

In one embodiment, an emulsion is formulated using a strontium salt and an amphiphilic polymer (e.g., PC-6). In another embodiment, a cream is formulated using a strontium salt and an amphiphilic polymer (e.g., PC-6). In another embodiment, a lotion is formulated using a strontium salt and an amphiphilic polymer (e.g., PC-6). In one embodiment, an emulsion is formulated using a strontium nitrate and an amphiphilic polymer (e.g., PC-6). In another embodiment, a cream is formulated using a strontium nitrate and an amphiphilic polymer (e.g., PC-6). In another embodiment, a lotion is formulated using a strontium nitrate and an amphiphilic polymer (e.g., PC-6). In one embodiment, an emulsion is formulated using a strontium chloride and an amphiphilic polymer (e.g., PC-6). In another embodiment, a cream is formulated using a strontium chloride and PC-6. In another embodiment, a lotion is formulated using a strontium chloride and an amphiphilic polymer (e.g., PC-6).

In one embodiment is a high concentration elemental strontium emulsion cream made with an amphiphilic polymer (e.g., PC-6). Emulsions are the mixture of two or more liquids that are normally immiscible, such as oil and water. In an emulsion, one liquid is dispersed in the other for example, oil can be dispersed in water or water can be dispersed in oil. Emulsifying agents are chemicals that stabilize the emulsion, that is, they help keep the dispersed liquid evenly distributed. Most emulsions would separate back into the two separate liquids without emulsifying agents.

In the process of developing the compositions disclosed herein, it was unexpectedly found that PC-6 behaves differently when using a strontium salt. For high salt formulations, e.g. over 5%, the manufacturer recommends using upwards of 5% of PC-6 to stabilize the formulation. Contrary to the manufacturer's recommendation, inventors surprisingly discovered that using less than 1% PC-6 was better for formulating high strontium salt emulsions. Without wishing to be bound by any one theory, it is believed that strontium's divalent cations synergistically interact with the PC-6 polymer in such a way as to balance the ionic interactions between the 3D spheres. Since one strontium cation can bind to two different 3D spheres, the strontium can assist in maintaining the dispersion of the spheres, similar to an emulsifying agent. Further, since the 3D spheres are hydrophilic on the interior, strontium cations can also be located in the interior of the spheres. Inventors surprisingly found that emulsion cream formulations containing 8% elemental strontium could be achieved using 0.5% PC-6. Based on the strontium to PC-6 ratio, inventors theorize that stable formulations containing even higher concentrations of elemental strontium could be achieved.

In one embodiment, a high concentration elemental strontium emulsion cream is made by hydrating the amphiphilic polymer (e.g., PC-6 polymer) in water. In another embodiment, the high concentration elemental strontium emulsion cream is made by hydrating the amphiphilic polymer (e.g., PC-6 polymer) in oil. Once the polymer is hydrated and has formed the 3D spheres, the strontium solution is dispersed in the polymer solution. The dispersion process also forces the strontium solution into the 3D spheres.

### Methods of Use

The above described compositions and formulations are designed to be topically applied. Topical application includes all epithelial surfaces, including but not limited to epidermis, keratinized epithelium, and mucous membranes in the eye, mouth, throat, esophagus, gastrointestinal tract, respiratory tract or genitourinary tract.

It is known that simple solutions of strontium and water can be effective at reducing pain and irritation when topically applied to skin. This indicates that strontium is able to pass through the outer layers of the skin without the inclusion of skin penetration enhancers. It is believed that one way in which strontium passes through the outer layers of the skin is through the use of pilosebaceous unit. The pilosebaceous unit made up of a hair follicle, hair shaft, and sebaceous gland. The follicle is about 1-4 micrometers in diameter. The epidermis involutes to form the interior of the follicle. However, the tough outer most layer of the epidermis, i.e. the stratum corneum is much thinner and/or non-existent within the follicle. Accordingly, compounds that are small enough to pass through the hair follicle can penetrate through the skin better than larger compounds. The ability of strontium to penetrate the skin through the use of the hair follicle reduces the need to add skin penetration enhancers to the formulation. While skin penetration enhancers are not necessary, in some cases, in certain embodiments it is beneficial to include a skin penetration enhancer to formulations of the present disclosure.

The strontium compositions described herein can be used to treat pain or pruritus associated with a variety of conditions. The conditions may be attributable to acute conditions such as allergies, insect bites, exposure to venom, poison ivy, atopic dermatitis, psoriasis, thermal burns, ionizing radiation, exposure to chemicals, trauma, surgery, nerve compression, oral or throat ulcers, bacterial infections, or viral infections. Alternatively, the conditions may be attributable to chronic conditions such as atopic dermatitis, psoriasis, viral infections, nerve compression, back pain, amputation, or trauma. In another alternative, the conditions may be attributable to neuropathic conditions such as post herpetic neuralgia, back pain, nerve compression, viral infections, multiple sclerosis, Parkinson's disease, diabetes, trauma, amputation, or drug use.

### EXAMPLES

### Example 1

### Exemplary High Concentration Strontium Emulsion

An exemplary high concentration elemental strontium based emulsion, e.g., having an elemental strontium concentration of 8-14%, is shown in Table 1 below.

TABLE 1

| | Amount |
|---|---|
| Ingredient | (% w/w) |
| Strontium salt | 15-40 |
| Xanthan gum | 0.1-0.5 |
| Glycerin | 1-5 |
| Potassium sorbate | 0.05-0.3 |
| Cetostearyl alcohol | 1-10 |
| Medium-chain triglycerides | 3-15 |
| polyOxyl 20 cetosteryl ether | 0.5-4 |
| Dimethicone | 0.1-2 |
| Polyacrylate crosspolymer-6 | 0.1-5 |
| Phenoxy ethanol | 0.5-3 |
| Hydrochloric acid 37% | To pH 4 ± 5 |
| Water | Balance |

### Example 2

### Formulation of an 8% Strontium Cream

An 8% strontium cream was made. In a first mixture, glycerin, xanthan gum, and polyacrylate crosspolymer-6 (PC-6) were combined. In a second mixture, water and potassium sorbate were combined. In a third mixture, polyOxyl 20 cetostearyl ether, dimethicone, cetostearyl alcohol, and triglycerides were combined. In a fourth compound, water and strontium were combined. The first and second mixtures were combined and heated to 75-80° C. The third mixture was heated to 75-80° C. and then added to the first two mixtures and mixed. The resulting mixture was cooled to less than 65° C., phenoxyethanol was added, and the mixture was cooled further to less than 50° C. The fourth mixture was then added to the first three mixtures and mixed at high speed. The resulting mixture was further cooled and the pH was adjusted to less than 4.5.

### Example 3

### Stability of an 8% Strontium Cream

The stability of the 8% elemental strontium cream from Example 1 was evaluated using standard conditions and accelerated conditions. The standard conditions essentially were 24 months at 25° C.±2° C. and 60%±5% relative humidity. The accelerated conditions were 6 months at 40° C.±2° C. and 75%±5% relative humidity. For the standard conditions, the samples were evaluated at three month intervals for appearance, odor, consistency, pH, microbial content, weight loss, viscosity, and amount of elemental strontium. For the accelerated conditions, the samples were evaluated every month for the same factors as the standard conditions. The results for the first six months are shown in Table 2 and 3 below.

TABLE 2
Standard Conditions

| Test | Specification | Initial | 3 month | 6 month |
|---|---|---|---|---|
| Appearance | Hazy to off-white | Conforms | Conforms | Conforms |
| Odor | No objectionable odor | Conforms | Conforms | Conforms |
| Consistency | Visually uniform | Conforms | Conforms | Conforms |
| | cream | | | |
| pH | 3.75 ± 0.5 | 3.43 | 3.49 | 3.45 |
| Microbial | <10 CFU/g | Not tested | Not tested | Not tested |
| Weight Loss | | 0.0% | 1.73% | 2.04% |
| Viscosity | | 3806 cP | 2228 cP | 2228 cP |
| Elemental | 8.0% ± 0.5% | 8.34% | 7.60% | 7.70% |
| Strontium | | | | |

TABLE 3
Accelerated Conditions

| Test | Specification | Initial | 3 month | 6 month |
|---|---|---|---|---|
| Appearance | Hazy to off-white | Conforms | Conforms | Conforms |
| Odor | No objectionable odor | Conforms | Conforms | Conforms |
| Consistency | Visually uniform | Conforms | Conforms | Conforms |
| | cream | | | |
| pH | 3.75 ± 0.5 | 3.43 | 3.55 | 3.35 |
| Microbial | <10 CFU/g | Not tested | Not tested | Not tested |
| Weight Loss | | 0.0% | 4.46% | 4.51% |
| Viscosity | | 3806 cP | 1966 cP | 1048 cP |
| Elemental | 8.0% ± 0.5% | 8.34% | 8.30% | 7.90% |
| Strontium | | | | |

### Example 4

### Modified Formulation Method of 8% Strontium Cream

An 8% strontium cream was made using a modified formulation method. In a first mixture, polyOxyl 20 cetostearyl ether, dimethicone, cetostearyl alcohol, and triglycerides were combined and heated to 75° C. until melted. Xanthan gum and PC-6 were added. In a second mixture, water, glycerin, and phenoxyethanol were combined and heated to 75° C. In a third mixture, water strontium nitrate and potassium sorbate were combined, heated to 45° C., and the pH adjusted to less than 4. The first and second mixtures were combined, homogenized, and cooled to 45° C. The third mixture was added to the first and second mixtures and homogenized.

### Example 5

### Stability of 8% Strontium Cream Using Modified Formulation Method

The stability of the 8% elemental strontium cream from Example 4 is evaluated using the standard conditions and accelerated conditions described in Example 3 above.

### Example 6

### Reduced Osmotic Shock

The osmotic shock of the 8% cream from Example 4 is evaluated against an 8% hydrogel. For each subject, the underside of the forearms are tape stripped to create microabrasions. The 8% cream is applied to one forearm and the 8% hydrogel is applied to the other forearm in a blinded manner. Subjects are asked to evaluate the itching, burning, stinging, or pain associated with each arm.

### Example 7

### Prolonged Release

The relief period of the 8% cream from Example 4 is evaluated against an 8% hydrogel. Subjects experiencing bi-lateral pain, itch, or irritation are used in the evaluation study. The 8% cream is applied to the affected area one side of the body and the 8% hydrogel is applied to the affected area on the other side of the body in a blinded manner. Subjects are asked to measure the length of time of relief from the pain, itch, or irritation is experienced for each side of the body.

### Example 8

### Acute Conditions

The ability of the 8% cream from Example 4 to reduce or eliminate acute pain, itch, or irritation is evaluated against a placebo cream. Subjects experiencing bi-lateral chronic pain, itch, or irritation are used in the evaluation study. The 8% cream is applied to the affected area on one side of the body and the placebo cream is applied to the affected area on the other side of the body. Subjects are asked to evaluate the pain, itch, irritation relief for each side of the body.

### Example 9

### Chronic Conditions

The ability of the 8% cream from Example 4 to reduce or eliminate chronic pain, itch, or irritation is evaluated against a placebo cream. Subjects experiencing bi-lateral chronic pain, itch, or irritation are used in the evaluation study. The 8% cream is applied to the affected area on one side of the body and the placebo cream is applied to the affected area on the other side of the body. Subjects are asked to evaluate the pain, itch, irritation relief for each side of the body.

### Example 10

### Neuropathic Conditions

The ability of the 8% cream from Example 4 to reduce or eliminate neuropathic pain, itch, or irritation is evaluated against placebo cream. Subjects experiencing bi-lateral neuropathic pain, itch, or irritation are used in the evaluation study. The 8% cream is applied to the affected area on one side of the body and the placebo cream is applied to the affected area on the other side of the body. Subjects are asked to evaluate the pain, itch, irritation relief for each side of the body.

### Example 11

### Reduction of Osmotic Shock-Induced Pain

Barrier damaged skin frequently occurs in acute and chronic pain, particularly in neuropathic pain and pruritic conditions, but can also occur in any form of skin inflammation or physical damage. Although the resulting stinging and burning pain is very transient and typically lasts only several seconds, it can be severe enough to cause a baby to cry if a product containing 2%, 4% or 6% elemental strontium is applied to barrier-damaged skin due to diaper rash. Similarly, if these products are applied to large areas of skin it can be sufficiently uncomfortable on inflammatory skin to cause a patient to stop using the product and thus not obtain its therapeutic benefits. Such irritation can trigger members of the Transient Receptor Potential (TRP) superfamily of receptors on keratinocytes, immune cells vascular endothelial cells and sensory nerves, including nociceptors that can cause long lasting exacerbation of the condition, especially in chronic conditions in which nociceptors are typically sensitized and have an exaggerated irritation and inflammatory response. Thus, it is desirable to reduce or eliminate such irritation due to the high osmolarity of the strontium in the composition, which potentially results in osmotic shock-induced irritation.

Formulations were prepared and tested for osmotic-shock induced irritation. Table 4 is a SepiMAX™ ZEN (polyacrylate crosspolymer-6)-based formulation containing 8% ‘elemental strontium’ (8% Sr+SepiMAX™ ZEN).

TABLE 4

| Component | % (W/W) |
|---|---|
| Sterile Water | 63.13 |
| Xanthan Gum | 0.3 |
| Glycerin | 2.5 |
| Potassium Sorbate | 0.15 |
| Cetostearyl Alcohol | 3.0 |
| Medium-chain Triglyceride | 8.0 |
| Polyoxyl 20 Cetostearyl Ether | 1.5 |
| Dimethicone | 0.5 |
| Polyacrylate Crospolymer-6 | 0.5 |
| Phenoxyethanol | 1.0 |
| Strontium nitrate anhydrous | 19.32 |
| Hydrochloric Acid (37%) | 0.1 |

The formulation as described in Table 4 was compared to a control formulation cream containing 4% elemental strontium.

Subjects were instructed to not apply any topical product to their face or legs for 24 hours prior to the test. Immediately prior to testing, subjects washed their faces or legs with Ivory non-scented bar cleanser and followed by thorough rinsing with warm tap water and drying. Subjects then shaved both sides of their face or the lateral portions of their calves, spanning from the ankle to below the knee using a disposable razor and ivory bar soap, followed by rinsing with tap water and air drying. The 4% Sr or 8% Sr formulations were randomly assigned to the right or left cheek or leg. Subjects then applied approximately 1 ml of a test material to an approximately 4 inch square skin area of one cheek or leg. For the next 5 minutes subjects recorded the maximum level of sensory irritation experienced using the following sensory irritation scale: 0=No stinging, burning, or itching; 1=Slight stinging, burning, or itching, barely detectable, but sensation is noticeable at rest, not bothersome; 2=Mild stinging, burning, or itching, definite sensation that would be detected even if the subject was not paying attention to it, bothersome; 3=Moderate stinging, burning, or itching, would always be distracting during daily routine, distinctly uncomfortable and bothersome; 4=Severe stinging, burning or itching, would interfere with daily routine, intolerable. The results of testing are presented in Tables 5 and 6.

TABLE 5

| | | Maximum Sensory Irritation Score | |
|---|---|---|---|
| Subject # | Location | 4% Sr | 8% Sr + SeppiMAX ™ ZEN |
| 1 | Face | 2 | 0 |
| 2 | Face | 3 | 1 |
| 3 | Face | 2 | 0 |
| 4 | Face | 2 | 0 |
| 5 | Face | 1 | 0 |
| 6 | Face | 2 | 0 |
| 7 | Face | 3 | 0 |
| 8 | Face | 3 | 1 |
| 9 | Face | 2 | 0 |
| 10 | Face | 2 | 0 |
| 11 | Face | 3 | 1 |
| 12 | Face | 2 | 0 |
| Mean | Face | 2.25 | 0.25 |

TABLE 6

| | | Maximum Sensory Irritation Score | |
|---|---|---|---|
| Subject # | Location | 4% Sr | 8% Sr + SeppiMAX ™ ZEN |
| 13 | Legs | 3 | 0 |
| 14 | Legs | 3 | 1 |
| 15 | Legs | 2 | 0 |
| Mean | Legs | 2.67 | 0.33 |

The 8% Sr and SeppiMAX™ ZEN containing formulation exhibited 89% less irritation of the face and 88% less irritation of the legs when compared to a 4% Sr formulation containing no SeppiMAX™ ZEN. These results demonstrated that the presence of the SepiMAX™ ZEN ingredient dramatically reduced the sensory irritation of barrier compromised skin, and that that the combination of strontium and SepiMAX™ ZEN unexpectedly dramatically reduced the osmotic shock-induced stinging and burning pain caused by strontium ions when applied to barrier damaged skin.

Barrier damaged skin frequently occurs in acute, chronic and specifically neuropathic pain and pruritic conditions and in any form of skin inflammation or physical damage, such that formulations containing both strontium and SepiMAX™ ZEN, even at elemental strontium concentrations as high as 8% will cause no or minimal irritation when applied to such barrier damaged skin, as shown in a model of barrier damaged skin due to the physical trauma of simple shaving. Such physical trauma closely resembles skin that has been scratched or broken due to inflammatory damage. In contrast, the application of strontium-containing products containing only 4% elemental strontium in the absence of an amphiphilic polymer such as SepiMAX™ ZEN caused highly uncomfortable levels of burning pain and stinging.

### Example 11

### PVP Cream Formulation

Strontium cream formulations were prepared by replacing SepiMAX™ ZEN (polyacrylate crosspolymer-6) in selected formulations as described above with polyvinylpyrrolidone (PVP), which was used as thickener and emulsion stabilizer. 1% PVP was dissolved in oil phase to achieve a stable emulsion in a presence of a high salt concentration. K-values assigned to various grades of PVP represent a function of the average molecular weight, the degree of polymerization, and the intrinsic viscosity. Formulations were prepared with PVP having K-values of 17, 30 and 90, as well as a formulation containing a commercial blend grade of PVP (available under the tradename FlexiThix™ from Ashland Inc.). Each of the formulations yielded cream formulas with stability both at ambient and high temperatures (40° C.) and through freeze-thaw cycles. The formulations also demonstrated stable pH values (around 3.8) during the stability testing. The viscosity of the cream formulations ranged from 6000 (initial) to 10000 cps (at equilibrium) for a PVP (K-17) formulation. The intrinsic viscosity of a PVP solution decreases due to intermolecular complex formation with cations. For a series of cations, the decreasing order of effectiveness in interaction of PVP in aqueous solutions, on the basis of molar concentrations, was found to be: K>Ca²⁺>Mg²⁺>Ba²⁺. PVP-containing formulations with equivalent strontium chloride salt were also made, although with different viscosities. Polyvinylpyrrolidone was effectively used as an emulsion stabilizer and thickener in the manufacturing of topical cream formulations containing strontium.

### Example 12

### PVP-Containing Formulations

A strontium nitrate topical cream formulation with 8% elemental strontium was prepared having a formula as in Table 7.

TABLE 7

| Component | % w/w |
|---|---|
| Water | 36.95% |
| Xanthan Gum | 0.30% |
| Glycerin | 2.50% |
| Potassium Sorbate | 0.15% |
| Phenoxyethanol | 1.00% |
| Capric/Caprylic Triglyceride | 8.00% |
| Cetostearyl Alcohol | 3.00% |
| Dimethicone | 1.00% |
| PVP | 1.00% |
| Ceteareth-20 | 1.50% |
| Water | 25.00% |
| Strontium Nitrate | 19.50% |
| Hydrochloric Acid (3.5) | 0.10% |

A strontium chloride hexahydrate topical cream formulation with 8% elemental strontium was prepared having a formula as in Table 8.

TABLE 8

| Component | % w/w |
|---|---|
| Water | 32.07% |
| Xanthan Gum | 0.30% |
| Glycerin | 2.50% |
| Potassium Sorbate | 0.15% |
| Phenoxyethanol | 1.00% |
| Capric/Caprylic Triglyceride | 8.00% |
| Cetostearyl Alcohol | 3.00% |
| Dimethicone | 1.00% |
| PVP | 1.00% |
| Ceteareth-20 | 1.50% |
| Water | 25.00% |
| Strontium Chloride Hexahydrate | 24.40% |
| Hydrochloric Acid (3.5) | 0.08% |

### Example 13

### Strontium Salt Formulations

A topical formulation containing 8% elemental strontium (from Strontium nitrate) was prepared having a formula as in Table 9.

TABLE 9

| Component | % w/w |
|---|---|
| Sterile Water | 63.13 |
| Xanthan Gum | 0.3 |
| Glycerin | 2.5 |
| Potassium Sorbate | 0.15 |
| Cetostearyl Alcohol | 3.0 |
| Medium-chain Triglyceride | 8.0 |
| Polyoxyl 20 Cetostearyl Ether | 1.5 |
| Dimethicone | 0.5 |
| Polyacrylate Crospolymer-6 | 0.5 |
| Phenoxyethanol | 1.0 |
| Strontium nitrate anhydrous | 19.32 |
| Hydrochloric Acid (37%) | 0.1 |

A topical formulation containing 6% elemental strontium (from strontium chloride) was prepared having a formula as in Table 10.

TABLE 10

| Component | % w/w |
|---|---|
| Sterile Water | 59.30 |
| Xanthan Gum | 0.3 |
| Glycerin | 2.5 |
| Potassium Sorbate | 0.15 |
| Cetostearyl Alcohol | 3.8 |
| Medium-chain Triglyceride | 11.5 |
| Polyoxyl 20 Cetostearyl Ether | 1.8 |
| Dimethicone | 0.8 |
| Polyacrylate Crospolymer-6 | 0.5 |
| Phenoxyethanol | 1.0 |
| Strontium chloride hexahydrate | 18.25 |
| Hydrochloric Acid (37%) | 0.1 |

Formulations were prepared using strontium chloride at 0, 4, 6, 8, 10 and 12% elemental strontium concentrations (C745 formulations) and using strontium nitrate at 0, 6, 8, 10, 12, 14% elemental strontium concentrations. All formulations were found to be stable through freeze-thaw cycles (3) and accelerated stability (40° C.) conditions without significant changes in the quality attributes of the formulations. The initial pH and viscosities for these formulations are provided in Table 11.

TABLE 11

| | Formulations Containing | | Formulations Containing | |
|---|---|---|---|---|
| Elemental | Strontium Chloride | | Strontium nitrate | |
| strontium | Viscosity | | Viscosity | |
| (%) | (cps) | pH | (cps) | pH |
| 4% | 14,500 | 3.5 | | |
| 6% | 22,000 | 3.5 | 15,200 | 3.4 |
| 8% | 21,700 | 3.8 | 13,000 | 3.5 |
| 10% | 23,800 | 3.7 | 13,900 | 3.4 |
| 12% | 15,400 | 4.0 | 23,000 | 2.6 |
| 14% | — | — | 14,300 | 3.5 |

### Example 14

### Usage of Various Polymers in Strontium Formulations

Strontium chloride formulations (6% elemental strontium) and Strontium nitrate formulations (8% elemental strontium) were prepared with 0.5% SepiMAX™ ZEN (Polyacrylate crosspolymer-6); 0.5% Pemulen TR-1 (Acrylates/C10-30 Alkyl Acrylate Crosspolymer); 0.5% Carbopol Ultrez30 (Carbomer, cross-linked homopolymer of acrylic acid); 1.5% Sepineo P600 (acrylamide/sodium acryloyldimethyl taurate copolymer); 1% Tego Carbomer 750 HD (Acrylates/C10-30 Alkyl Acrylate Crosspolymer) polymers. The initial pH and viscosities observed for these formulations are presented in Table 12.

TABLE 12

| | | Strontium chloride | | Strontium nitrate | |
|---|---|---|---|---|---|
| Formulations | | (6% elemental strontium) | | (8% elemental strontium) | |
| SepiMAX ™ | 0.5% | 22,000 cps | 3.5 | 16,100 cps | 2.7 |
| ZEN | | | | | |
| Pemulen | 0.5% | 18,000 cps | 3.1 | 26,200 cps | 3.0 |
| TR-1 | | | | | |
| Carbopol | 0.5% | 11,000 cps | 3.5 | 14,300 cps | 3.5 |
| Ultrez30 | | | | | |
| Sepineo | 1.5% | 15,600 cps | 2.6 | 14,400 cps | 2.9 |
| P600 | | | | | |
| Carbomer | 1.0% | 38,300 cps | 3.1 | 31,200 cps | 2.9 |
| 750 HD | | | | | |

### Example 15

### Impact of SepiMAX™ ZEN Concentrations

Strontium chloride formulations (6% elemental strontium) were prepared using 0, 0.5, 1, 2 and 3% SepiMAX™ ZEN polymer content. Strontium nitrate formulations (8% elemental strontium) were prepared with 0, 0.5, 1 and 3% SepiMAX™ ZEN polymer content. The formulations resulted in viscosities ranging from 13,000 to 70, 000 cps. 3% SepiMAX™ ZEN containing formulas typically gave a higher viscosity of about 70, 000 cps. All formulations were stable through freeze-thaw cycles and accelerated stability conditions. An increase in the viscosity of high salt formulations was observed with an increase in the SepiMAX™ ZEN concentration, as shown in Table 13.

TABLE 13

| | Strontium Chloride | | Strontium nitrate | |
|---|---|---|---|---|
| | Formulations | | Formulations | |
| SepiMAX ™ | (6% elemental Strontium) | | (8% elemental Strontium) | |
| ZEN | Viscosity | | Viscosity | |
| (%) | (cps) | pH | (cps) | pH |
| 0 | 25,600 | 3.8 | 12,200 | 3.3 |
| 0.5 | 22,000 | 3.5 | 13,000 | 3.5 |
| 1.0 | 25,700 | 3.5 | 12,900 | 3.3 |
| 2.0 | 29,500 | 2.4 | | |
| 3.0 | 70,500 | 3.6 | 61,200 | 3.0 |

### Example 16

### Impact of Xanthan Gum on the Formulations

Strontium topical formulations also containing Xanthan Gum as an additional viscosity agent were prepared. Strontium chloride formulations (6% elemental strontium) were prepared using 0.1, 0.3 and 0.8% Xanthan gum concentrations. The formulations resulted in viscosities ranging from 14,000 to 22,000 cps, as shown in Table 14.

TABLE 14

| Xanthan Gum | Viscosity |
|---|---|
| (%, W/W) | (cps) |
| 0.1 | 14,200 |
| 0.3 | 22,000 |
| 0.8 | 21,100 |

### Example 17

### Impact of Oil/Water Ratio on the Viscosity of Strontium Formulations

Topical formulations containing strontium salts were prepared with oil phase consists of Capric caprylic triglyceride, cetostearyl alcohol, cetereth-20 and Dimethicone. Ratios of water and oil phases were studied to adjust the viscosity of these formulations, as shown in Table 15.

TABLE 15

| | Strontium chloride | Strontium nitrate |
|---|---|---|
| Oil/water | formulation | formulation |
| Ratio | (6% elemental strontium) | (8% elemental strontium) |
| 0.15 | 6,400 cps | 11,500 cps |
| 0.21 | 10,000 cps | 13,000 cps |
| 0.24 | 19,600 cps | 12,500 cps |
| 0.27 | 22,000 cps | |
| 0.3 | 21,000 cps | 23,300 cps |
| 0.35 | 15,200 cps | |
| 0.4 | 13,200 cps | |
| 0.45 | 18,600 cps | |

### Example 18

### Impact of pH on the Formulations

Strontium topical formulations were developed to be in acidic pH by design. The impact of the pH on the formulations was studied by adjusting the process pH, as shown in Table 16.

TABLE 16

| Initial | Viscosity |
|---|---|
| pH | (cps) |
| 2.1 | 14,000 |
| 3.8 | 19,600 |
| 4.1 | 21,200 |
| 4.2 | 28,000 |

### Example 19

### Impact of Octyl Palmitate on Topical Formulations Containing Strontium Salts

Capric caprylic triglyceride comprised a majority (about 70%) of the oil phase in the strontium salt formulations of other examples. Octyl Palmitate was used to replace Capric caprylic triglyceride in the formulations and the effect on viscosity was studied, as shown in Table 17.

TABLE 17

| Oil/Water | Formulations with | Formulations with |
|---|---|---|
| Ratio | Capric Caprylic triglyceride | Octyl Palmitate |
| 0.24 | 19,600 cps | 19,300 cps |
| 0.27 | 22,000 cps | 13,600 cps |
| 0.3 | 14,000 cps | 29,300 cps |

### Example 20

### Exemplary Strontium Formulation

Table 18 provides composition data for an exemplary strontium formulation.

TABLE 17

| INCI/USP Ingredients | CAS No. | Quality/Grade | % w/w |
|---|---|---|---|
| Water/Purified Water | 7732-18-5 | Deionized or | 60.80 |
| | | Purified | |
| Strontium Nitrate | 10042-76-9 | 99% pure, -35mesh | 9.70 |
| Isopropyl Myristate | 110-27-0 | Technical | 7.50 |
| Butyrospermum Parkii | 68920-03-6 | Deodorized/ | 3.50 |
| (Shea) Butter | | Technical | |
| Caprylic/Capric | 73398-61-5 | Technical | 3.50 |
| Triglyceride | | | |
| Glycerin | 56-81-5 | USP | 3.00 |
| Cetearyl Alcohol/ | 67762-27-0 | Technical | 3.00 |
| Cetostearyl Alcohol | | | |
| Emulsifying Wax NF | 67762-27-0; | USP | 3.00 |
| | Proprietary | | |
| Glyceryl Stearate | 67701-33-1 | Technical | 1.00 |
| Magnesium Aluminum | 12199-37-0 | Technical | 1.00 |
| Silicate | | | |
| Dimethicone | 63148-62-9 | Technical | 1.00 |
| Phenoxyethanol | 122-99-6 | Technical | 1.00 |
| Polyacrylate | 1190091-71-4 | Technical | 0.80 |
| Crosspolymer-6 | | | |
| Xanthan Gum | 11138-66-2 | NF; Eur. Ph | 0.50 |
| Caprylyl Glycol | 1117-86-8 | Technical | 0.30 |
| Ceramide | 2304-80-5 | Technical | 0.10 |
| Sodium Hyaluronate | 9067-32-7 | Technical | 0.10 |
| Disodium EDTA | 139-33-3 | USP | 0.10 |
| Tetrahexyldecyl | 183476-82-6 | Technical | 0.05 |
| Ascorbate | | | |
| Tocopherol | 10191-41-0 | USP | 0.05 |

While the disclosure has been illustrated and described in detail in foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single component may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A topical formulation, comprising:
from 8 wt. % to 12 wt. % elemental strontium in a form of a strontium moiety; and
from 0.5 wt. % to 1.5 wt. % of a polyacrylate crosspolymer-6 amphiphilic polymer,
wherein the topical formulation has a pH of 2 to 5, and a hydrophilic-lipophilic balance of about 9 to about 11, wherein the hydrophilic-lipophilic balance is defined as 20×Mₕ/M, where Mₕ is defined as a molecular mass of a hydrophilic portion of the polymer, and M defined as is a molecular mass of the polymer as a whole.

2. The topical formulation of claim 1, wherein the polyacrylate crosspolymer-6 amphiphilic polymer is 70% to 80% hydrophobic and has a hydrophilic tail.

3. The topical formulation of claim 1, wherein the polyacrylate crosspolymer-6 amphiphilic polymer forms three dimensional structures in the topical formulation that are roughly spherical or like a flattened sphere in shape, the three dimensional structures having an exterior and an interior, wherein the exterior is mainly hydrophilic and the interior is mainly hydrophilic, and wherein a diameter of the three dimensional structures is less than 2 micrometers.

4. The topical formulation of claim 1, having a viscosity of 1000 centipoise to 25000 centipoise.

5. The topical formulation of claim 1, having a viscosity of 1000 centipoise to 10000 centipoise.

6. The topical formulation of claim 1, wherein the strontium moiety is a strontium salt selected from the group consisting of strontium nitrate, strontium chloride, strontium chloride hexahydrate, strontium hydroxide, and strontium lactate.

7. The topical formulation of claim 1, wherein the strontium moiety is strontium nitrate or strontium chloride.

8. A method of manufacturing a topical formulation, comprising:
wetting a polyacrylate crosspolymer-6 polymer in an oil phase;
dissolving a strontium moiety into an aqueous phase; and
mixing at high speed the wetted polyacrylate crosspolymer-6 polymer and the dissolved strontium moiety to create a stable emulsion, whereby a topical formulation is obtained, the topical formulation comprising from 8 wt. % to 12 wt. % elemental strontium in a form of the strontium moiety, and from 0.5 wt. % to 1.5 wt. % of the polyacrylate crosspolymer-6 amphiphilic polymer, wherein the topical formulation has a pH of 2 to 5, and a hydrophilic-lipophilic balance of about 9 to about 11, wherein the hydrophilic-lipophilic balance is defined as 20×Mₕ/M, where Mₕ is defined as a molecular mass of a hydrophilic portion of the polymer, and M defined as is a molecular mass of the polymer as a whole.

9. The method of claim 8, wherein the polyacrylate crosspolymer-6 polymer is mainly hydrophobic.

10. A method of treating peripheral neuropathic pain, comprising:
administering to a patient in need thereof a therapeutically effective amount of the topical formulation of claim 1.

11. The method of claim 10, wherein the pain is due to a condition selected from the group consisting of an insect bite, a thermal burn, an ionizing radiation, an exposure to a chemical, a surgical wound, a nerve compression, or a viral infection.

12. The method of claim 11, wherein the viral infection is due to Herpes varicella zoster virus.

13. The method of claim 11, wherein the viral infection is due to human immunodeficiency virus.

14. The method of claim 10, wherein the pain is due to a condition selected from the group consisting of post herpetic neuralgia, diabetic neuropathy, or a side effect of use of a drug.

15. The method of claim 14, wherein the drug is used to treat HIV.
